SEQUENCE LISTING (39 sequences)

SEQ ID NO: 1 - protein, 213 aa, Artificial Sequence
  A synthetic sequence
MTAPFTHGPLRVWSLPTGPIQENAVLIAGEQGQGFLIDPGDDAGRIAALVAASGVTVTGILLTHAHFDHIGAVQPLREQLGVPVWLHPDDRELYALGAQSAARWNLPFTQPAPPDHDITGGQTFTAGDLTLTARHLPGHAPGHVVFVAPGVVIAGDTLFQGGIGRTDLPGGNHPQLLAGIRTQLLTLPDDTAVYPGHGPRTSVGHERRSNPFL

SEQ ID NO: 2 - protein, 281 aa, Artificial Sequence
  A synthetic sequence
MSWNHTRQIGQAQVHSLTDGQFRLDGGAMFGSVPKALWERAAPADDLNRIRLRINPLLIQLGGENILVETGFWDQGGEKFEGMYALDRDETVFRGLDRLGLSPEDIHLVINTHLHFDHAGRNVTLLGDPTFPNARYVVQKQELHDARHTHERSRASYIPAYIDPILDAGLFDVVDGEHELRPGLSVLPLPGHNLGQQGVVLRSEGQTLVYVADLIPTLAHAPTPYIMGYDLYPVTTLETRKAHLGAWFEQNATICTPHDPDAPFARLHENPKGGFTLQADS

SEQ ID NO: 3 - protein, 281 aa, Artificial Sequence
  A synthetic sequence
MSWNHSRQIGQAQVHSLTDGQFRLDGGAMFGSVPRVLWERAAPADDLNRIRLRINPLLIQLGGENILVETGFWDQGGEKFEGMYALDRDETVFRGLDRLGLSPEDIHLVINTHLHFDHAGRNVTLLGDPTFPNARYVVQKQELHDARHTHERSRASYIPAYIDPILDAGLFDVVDGEHELRPGLSVLPLPGHNLGQQGVVLRSEGQTLVYVADLIPTLAHAPTPYIMGYDLYPVTTLETRKAHLGAWFEQNAIICTPHDPDAPFARLHENPKGGFTLQADS

SEQ ID NO: 4 - protein, 281 aa, Artificial Sequence; identical to SEQ ID NO: 3
  A synthetic sequence

SEQ ID NO: 5 - protein, 240 aa, Artificial Sequence
  A synthetic sequence
MKRLGDVIVLELPATLMGTPSVIHPVALVGPDHILTLVDTGLPGMLDAISGELHAADFTLGQVRRVIVTHHDLDHIGSLEAVVHATGAEVWALEPEVPYVTGERRAQKLPSPEQAQAMLADPDLNPTMRAMLTRDPVRVPVSRALRDGDLLPGQVRVIATPGHTPGHLSLLVPGGNILISGDALTSQDGALHGPLSRATPDLPGAHDSVRRLAQEDVQTIVTYHGGVVSDDAGGQLRALA

SEQ ID NO: 6 - protein, 240 aa, Artificial Sequence
  A synthetic sequence
MKRLGDVIVLELPATLMGTPSVIHPVAPVGPDHILTLVDTGLPGMLDAISGELHAADFTLGQVRRVIVTHHDLDHIGSLEAVVHATGAEVWALEPEVPYVTGERRAQKLPSPEQAQAMLQEPDLNPVMRALLTREPVRVPVSRALRDGDLLPGQVRVIATPGHTPGHLSLLVPGGNILISGDALTSQDGALHGPIPRATPDLPGAHASVRRLAQEDVQTIVTYHGGVVSDAAGGQLRALA

SEQ ID NO: 7 - protein, 245 aa, Artificial Sequence
  A synthetic sequence
MKRLGDVIVLELPATLMGTPSVIHPVALVGPDHILTLVDTGLPGMLDAIISELHAADFTLGQVRRVIVTHHDLDHIGSLEAVVHATGAEVWALEPEVPYVTGERRAQKLPSPEQAQAMLADPDLNPTMRALLTREPVRVPVSRALRDGDLLPGQVRVIATPGHTPGHLSLLVPGGNILISGDALTAQGGMLRGPIPRATPDLPGAHDSVRRLAQEDVQTIVTYHGGVVSDDAGGQLRALAASLDS

SEQ ID NO: 8 - protein, 240 aa, Artificial Sequence
  A synthetic sequence
MKRLGDVIVLELPATLMGTPSVIHPVALVGPDHILTLVDTGLPGMLDAIIGELHAADFTLGQVRRVIVTHHDLDHIGSLESVVHATGAEVWALEPEVPYVTGERRAQKLPSPEQAQAILADPDLNPATRALLTREPTRVPVSRALRDGDLLPGHVRVIATPGHTPGHLSLLVPGGNILISGDALTSQDGALHGPLSRATPDLPGAHASVRRLAQEDVQTIVTYHGGVVSDDAGGQLRALA

SEQ ID NO: 9 - protein, 270 aa, Artificial Sequence
  A synthetic sequence
VSVRVIPLRAGSCLNLAAITERGAPWRVQAYPAGFTLILHPTRGPVLFDTGYGADVLTAMRRWPGVIYGLITPVQFGPHDSAHEQLRVMGFPPKEVRHIIVSHLHADHVGGLRDFPHATFHLDRRAWEPLRALRGVRAVRRAYLPELLPDDFEDRCTWLDFKEAGNALHPFAEVADVFGDGLLRAVPLPGHAPGMVGILAQEDAGLTVLAADAAWSVRAGREERPVHPLARVAFHDPAQEATSGAALRAFLHANPGARLHVSHDAPEGWT

SEQ ID NO: 10 - protein, 270 aa, Artificial Sequence
  A synthetic sequence
MSVRVVPLWAGSCLNLSAITERGAPWRVQAYPAGFTLILHPTRGPVLFDTGYGADVLTAMRRWPGLIYGLITPVQFGPHDSAREQLRVLGFPPKEVRHIIVSHLHADHVGGLRDFPHATFHLDRRAWEPLRALRGVRAVRRAYLPELLPDDFEDRCTWLDFKEAGNALHPFAEVADVFGDGLLRAVPLPGHAPGMVGILAQEDAGLTVLAADAAWSVRAGREERPVHPLARVAFHDPAQEAASGAALRAFLHANPGARLHVSHDAPEGWT

SEQ ID NO: 11 - protein, 270 aa, Artificial Sequence
  A synthetic sequence
VSVRVIPLRAGSCLNLAAITERGAPWRVQAYPAGFTLILHPTRGPVLFDTGYGADVVTAMRRWPGVIYGLITPVQFGPHDSAHEQLRVMGFPPEEVRHIIVSHLHADHVGGLRDFPHATFHLDRRAWEPLRALRGVRAVRRAYLPELLPDDFEDRCTWLDFKEAGNALHPFAEVADVFGDGLLRAVPLPGHAPGMVGLLAQEEAGLTVLAADAAWSVRAGREERPVHPLARVAFHDPAQEATSGAALRAFLHANPAARLHVSHDVPEGWT

SEQ ID NO: 12 - protein, 323 aa, Artificial Sequence
  A synthetic sequence
MSAQTVTGPVPASKLGFTLPHEHVLFGYPGYQGDLTLGPFDREAALNACEDVARSLLSRGVRTLVDATPNECGRDPAFLRDLSERSGLRILCSSGYYYEGEGAATYFKFRASLGGGEAEIEELMRHEVTVGIGSSGVRAGVIKLASSRDAITPYEQMFFRAAARVQRDTGVPIITHTQEGRQGPQQAQLLLSHGADPARIMIGHMDGNTDPAYHRETLSHGVSVAFDRLGLQGLVGTPTDAQRLDVLTTLLGEGFADRILLSHDSIWQWLGRPIPMPDAILGAVKDWHPLHLTDDILPELERRGVGAEQLRQMTVGNPARLFG

SEQ ID NO: 13 - protein, 294 aa, Artificial Sequence
  A synthetic sequence
MMMAAGLHVGARAQGTTTAALTNGAGFYRFKLGDFTCMVISDGQSTGGNTFPNWGANPGRQEEFGKVLQANFIPIEPFTNNFNPMVIDTGKNKVLIDTGRGGTNGQLLQNLRNAGLTPADIDTVFITHGHGDHIGGMTDAAGASVFANAKLVMGQQEFDFWASQNNAGFNRNIVPFKDRFTFVKDGDEIVPGLTAVATPGHTAGHMAVLATSGTNKLMHFGDAGGHFLLSLMFPDHYLGFDSNPENATATRKKIFEMAANERMMVVGYHYAWPGVGNIRKKDAAYEFVPTFFRF

SEQ ID NO: 14 - protein, 323 aa, Artificial Sequence
  A synthetic sequence
MSAQTVTGPVPASELGFTLPHEHVLFGYPGYQGDLTLGPFDREAALSVCEDVARSLLARGVRTLVDATPNECGRDPAFLRDLSERSGLRILCSSGYYYEGEGAATYFKFRASLGGGEAEIEELMRHEVTVGIGSSGVRAGVIKLASSRDAITPYEQMFFRAAARVQRDTGVPIITHTQEGRQGPQQAQLLLSHGADPARIMIGHMDGNTDPAYHRETLSHGVSVAFDRLGLQGLVGTPTDAQRLDVLTTLLGEGFADRILLSHDSIWQWLGRPIPMPDAILGAVKDWHPLHLTDDILPELERRGVGAEQLRQMTVGNPARLFG

SEQ ID NO: 15 - protein, 762 aa, Pseudomonas aeruginosa
MGMRTVLTGLAGMLLGSMMPVQADMPRPTGLAADIRWTAYGVPHIRAKDERGLGYGIGYAYARDNACLLAEEIVTARGERARYFGSEGKSSAELDNLPSDIFYAWLNQPEALQAFWQAQTPAVRQLLEGYAAGFNRFLREADGKTTSCLGQPWLRAIATDDLLRLTRRLLVEGGVGQFADALVAAAPPGAEKVALSGEQAFQVAEQRRQRFRLERGSNAIAVGSERSADGKGMLLANPHFPWNGAMRFYQMHLTIPGRLDVMGASLPGLPVVNIGFSRHLAWTHTVDTSSHFTLYRLALDPKDPRRYLVDGRSLPLEEKSVAIEVRGADGKLSRVEHKVYQSIYGPLVVWPGKLDWNRSEAYALRDANLENTRVLQQWYSINQASDVADLRRRVEALQGIPWVNTLAADEQGNALYMNQSVVPYLKPELIPACAIPQLVAEGLPALQGQDSRCAWSRDPAAAQAGITPAAQLPVLLRRDFVQNSNDSAWLTNPASPLQGFSPLVSQEKPIGPRARYALSRLQGKQPLEAKTLEEMVTANHVFSADQVLPDLLRLCRDNQGEKSLARACAALAQWDRGANLDSGSGFVYFQRFMQRFAELDGAWKEPFDAQRPLDTPQGIALDRPQVATQVRQALADAAAEVEKSGIPDGARWGDLQVSTRGQERIAIPGGDGHFGVYNAIQSVRKGDHLEVVGGTSYIQLVTFPEEGPKARGLLAFSQSSDPRSPHYRDQTELFSRQQWQTLPFSDRQIDADPQLQRLSIRE

SEQ ID NO: 16 - protein, 223 aa, Pseudomonas aeruginosa
PRPTGLAADIRWTAYGVPHIRAKDERGLGYGIGYAYARDNACLLAEEIVTARGERARYFGSEGKSSAELDNLPSDIFYAWLNQPEALQAFWQAQTPAVRQLLEGYAAGFNRFLREADGKTTSCLGQPWLRAIATDDLLRLTRRLLVEGGVGQFADALVAAAPPGTEKVALSGEQAFQVAEQRRQRFRLERGSNAIAVGSERSADGKGMLLANPHFPWNGAMRF

SEQ ID NO: 17 - protein, 779 aa, Pseudomonas aeruginosa
MIISRPLCGFVFAGLSFAVILPAQALVAADNQAARAEIRRTGFGVPHIVAANERGLGYGIGYAYAQDNLCLLANEVVTVNGERSRYFGPDKATLEQRSNMASDLLFKWLNTPEALADFWKAQPPEIRQLMQGYVAGYNRSLDEQKTKGLPRPCAADWVRPISTDDLLRLTRRLLVEGGVGQFTEAFAGAKPPSAQKPLQVDSQQVQALQLAAVRNQRFALERGSNAVAVGHELSANGRGMLLANPHFPWGGGMRFYQMHLTIPGKLDVMGAALPGLPLINIGFNRHLAWSHTVDTSKHFTLHRLQLDPKDSTRYLLDGKSIAMGQQQVSVEVKQPDGSLKDVPRIIYSSKFGPVVQWPGKLDWDDKFAFSLRDANLENDRVLQQWYSMDKADSLKAFQDSLHKIQGIPWVNTLAVDAKGQALYMNLSVVPNVDAAKLAKCSDPRIGTELIVLDGSRSECNWDISAEAAQAGIYPSSRQPQLLRSDFVQHSNDSAWMVNPAAPLKGFSPLISQDGQPLGQRARFALDRLGSLQQAGKVSAENLQAMVMDNEVYQAGQVLPDLLKFCASELGDDVARLTPLCAALKAWDGRADLNSGIGFVYFQRIMTSMQGVASRWRVVFDPQNPIHTPSGLAIENPQVASALRAAMLAAVDEVAKAGLSPESKWGDIQVSSLSGKPIPIHGGPAGLGVYNAMQTIAGKDGKREVVSGTSYLQVVTFDEQGPRAQGLLAFSESSNPQSAHSSDQTEAFSKKQWSELPFTEQQIKADPAYQVQVISEEGSR

SEQ ID NO: 18 - protein, 459 aa, Pseudomonas ficuserectae
MAMGQQQVSVEVKQPDGSLKAVPRIIYSSKFGPVVQWPGKLDWDDKFAFSLRDANLENDRVLQQWYSMDKADSLKAFQDSVHKIQGIPWVNTLAVDAKGQALYMNLSVVPNVDAAKLAKCSDPRIGTELIVLDGSRSECNWDISAEAAQAGIYPSSRQPQLLRSDFVQHSNDSAWMVNPAAPLKGFSPLISQDGQPLGQRARFALDRLGSLQQAGKVSAENLQAMVMDNEVYQAGQVLPDLLKFCASELGDDVARLTPLCAALKAWDGRADLNSGIGFVYFQRIMTSMQGVASRWRVVFDPQNPIHTPSGLAIENPQVASALRAAMLAAVDEVAKAGLSPESKWGDIQVSSLSGKPIPIHGGPAGLGVYNAMQTIAGKDGKREVVSGTSYLQVVTFDEQGPRAQGLLAFSESSNPQSTHSSDQTEAFSKKQWSELPFTEQQIKADPAYQVQVISEEGSR

SEQ ID NO: 19 - protein, 250 aa, Bacillus thuringiensis
MTVKKLYFIPAGRCMLDHSSVNSTLAPGNLLNLPVWCYLLETEEGPILVDTGMPESAVNNEGLFNGTFVEGQILPKMTEEDRIVNILKRVGYEPDDLLYIISSHLHFDHAGGNGAFTNTPIIVQRTEYEAALHREEYMKECILPHLNYKIIEGDYEVVPGVQLLYTPGHSPGHQSLLIETEKSGPVLLTIDASYTKENFEDEVPFAGFDSELALSSIKRLKEVVMKEKPIIFFGHDIEQEKGCKVFPEYI

SEQ ID NO: 20 - protein, 250 aa, Bacillus thuringiensis
MTVKKLYFIPAGRCMLDHSSVNSTLAPGNLLNLPVWCYLLETEEGPILVDTGMPESAVNNEGLFNGTFVEGQILPKMTEEDRIVNILKRVGYEPDDLLYIISSHLHFDHAGGNGAFTNTPIIVQRTEYEAALHREEYMKECILPHLNYKIIEGDYEVVPGVQLLYTPGHSPGHQSLLIETEKSGPVLLTIDASYTKENFEDEVPFAGFDSELALSSIKRLKEVVMKEKPIIFFGHDIEQEKGFKVFPEYI

SEQ ID NO: 21 - protein, 250 aa, Bacillus thuringiensis
MTVKKLYFIPAGRCMLDHSSVNSALTPGKLLNLPVWCYLLETEEGPILVDTGMPERAVNNEGLFNGTFVEGQILPKMTEEDRIVNILKRVGYEPDDLLYIISSHLHFDHAGGNGAFTNTPIIVQRTEYEAALHREEYMKECILPHLNYKIIEGDYEVVPGVQVLYTPGHSPGHQSLFIETEQSGSVLLTIDASYTKENFEDEVPFAGFNPELALSSIKRLKEVVAKEKPIVFFGHDIEQEKGCRVFPEYI

SEQ ID NO: 22 - protein, 323 aa, Rhodococcus erythropolis
MSSVQTVRGPVDTADLGKVLMHEHVFVLGEELRQNYPDYPEPWDEEVRVADAVAKLTEVKSRGISTIVDPTVIGLGRYIPRIVRINEQVDLNIIVATGIYTYNDIPFQFHYTGPGLLFDGPEPMVELFVKDIREGIAGTGVRASFLKCAIEEPGLTPGVERVMRAVGQSQVETGVPITVHTNPHTESGLVAQKVLAEEGADLTKVVIGHSGDTTDLDYLCALADAGSLLGMDRFGLEPFLSFEDRVNTVVEMARRGYAEKMVLAHDASCFIDYFPSAAREVALPNWNYTHISDDVIPALLERGVTEDQVKAMLVDNPRRYFES

SEQ ID NO: 23 - protein, 323 aa, Rhodococcus 420
MSSVQTVRGPVDTADLGKVLMHEHVFVLGEEMRQNYPDYPEPWDEEARVADAVTKLSELKSRGISTIVDPTVIGLGRYIPRIARINEQVDLNIIVATGVYTYNDIPFQFHYTGPGLLFDVREPMVDLFVKDIKGGIAGTDIRASFLKCAIEEPGLTPGVERVMRAVGQAQVETGVPITVHTNPHTESGLVAQKVLAEEGADLSKVVIGHSGDSTDLDYLCALADAGSLLGMDRFGLDVLLPFEERVNTVVELARRGYAEKMVLAHDASCFIDWFSSEAKAAAVPNWNFTHISDQVIPTLLERGVTEDQINTMLVDNPRRYFES

SEQ ID NO: 24 - protein, 323 aa, Rhodococcus Chrous; identical to SEQ ID NO: 22

SEQ ID NO: 25 - protein, 281 aa, Artificial Sequence; identical to SEQ ID NO: 2
  A synthetic sequence

SEQ ID NO: 26 - protein, 281 aa, Artificial Sequence; identical to SEQ ID NO: 3
  A synthetic sequence

SEQ ID NO: 27 - protein, 281 aa, Artificial Sequence; identical to SEQ ID NO: 3
  A synthetic sequence

SEQ ID NO: 28 - protein, 240 aa, Artificial Sequence; identical to SEQ ID NO: 8
  A synthetic sequence

SEQ ID NO: 29 - protein, 245 aa, Artificial Sequence; identical to SEQ ID NO: 7
  A synthetic sequence

SEQ ID NO: 30 - protein, 240 aa, Artificial Sequence; identical to SEQ ID NO: 5
  A synthetic sequence

SEQ ID NO: 31 - protein, 240 aa, Artificial Sequence; identical to SEQ ID NO: 6
  A synthetic sequence

SEQ ID NO: 32 - protein, 270 aa, Artificial Sequence; identical to SEQ ID NO: 10
  A synthetic sequence

SEQ ID NO: 33 - protein, 270 aa, Artificial Sequence; identical to SEQ ID NO: 9
  A synthetic sequence

SEQ ID NO: 34 - protein, 270 aa, Artificial Sequence; identical to SEQ ID NO: 11
  A synthetic sequence

SEQ ID NO: 35 - protein, 323 aa, Artificial Sequence; identical to SEQ ID NO: 14
  A synthetic sequence

SEQ ID NO: 36 - protein, 323 aa, Artificial Sequence
  A synthetic sequence
MSAQTVTGPVPASKLGFTLPHEHVLFGYPGYQGDLTLGPFDREAALNACEDVARSLLSRGMSAQTVTGPVPASKLGFTLPHEHVLFGYPGYQGDLTLGPFDREAALNACEDVARSLLSRGEELMRHEVTVGIGSSGVRAGVIKLASSRDAITPYEQMFFRAAARVQRDTGVPIITHTQEGRQGPQQAQLLLSHGADPARIMIGHMDGNTDPAYHRETLSHGVSVAFDRLGLQGLVGTPTDAQRLDVLTTLLGEGFADRILLSHDSIWQWLGRPIPMPDAILGAVKDWHPLHLTDDILPELERRGVGAEQLRQMTVGNPARLFG

SEQ ID NO: 37 - protein, 294 aa, Artificial Sequence; identical to SEQ ID NO: 13
  A synthetic sequence

SEQ ID NO: 38 - protein, 327 aa, Artificial Sequence
  A synthetic sequence
MSDHTPTDDRPTPTLARRDALRLIGTAGALAAAAPLARAQTAPAAPAAMAPMNGNGFYRQKIGDMTVTVFSDGTAPLAALLPTWGANPDRQAEFTQTLAEYHVAAANTVNHFNPVLVEQGGQRILIDTGRGGANGQLVANMRRAGIEPDSVNTVFITHGHGDHIGGLTTAGKPTFANAQHVMGEAEFNFWVTQANPNDAVKNNLIALKDRFRLIAPGQEIVPGLTSVPTPGHTANHLSVLAQSGGQGIMILGDAGGHFLISLKHQGAYVGFDTNGALAAQTRQRIFNRIVTEGLWVTGYHFPFHAIGHLRRLNARSYEYEPTLWNWS

SEQ ID NO: 39 - protein, 213 aa, Artificial Sequence; identical to SEQ ID NO: 1
  A synthetic sequence

(19) United States
(12) Patent Application Publication
(10) Pub. No.: US 2022/0411769 A1
(43) Pub. Date: Dec. 29, 2022

(54) METHODS OF USING BACTERIAL QUORUM QUENCHING ENZYMES

(71) Applicants: Sabrina Robin Mueller-Spitz, Oshkosh, WI (US); Kevin Douglas Crawford, Oshkosh, WI (US); Christopher M. Bianchetti, Middleton, WI (US)

(72) Inventors: Sabrina Robin Mueller-Spitz, Oshkosh, WI (US); Kevin Douglas Crawford, Oshkosh, WI (US); Christopher M. Bianchetti, Middleton, WI (US)

(21) Appl. No.: 17/843,552

(22) Filed: Jun. 17, 2022

Related U.S. Application Data
(60) Provisional application No. 63/212,364, filed on Jun. 18, 2021.

(51) Int. Cl. C12N 9/14; C12N 15/82; C12N 15/74

(52) U.S. Cl. CPC C12N 9/14; C12N 15/8202; C12N 15/8216; C12N 15/8241; C12N 15/74

(57) ABSTRACT

A method to prevent, inhibit or treat soft rot in a vegetable, fruit or ornamental plant is provided, as well as compositions comprising one or more isolated quorum quenching lactonases.

### CROSS-REFERENCE TO RELATED APPLICATIONS

This application claims the benefit of the filing date of U.S. application No. 63/212,364, filed on Jun. 18, 2021, the disclosure of which is incorporated by reference herein.

### BACKGROUND

One way to improve the sustainability of global food production systems is to minimize waste and loss in the process. It has been estimated that about 45% of fruits and vegetables are lost before making it to consumers, contributing to food security issues (Graz University of Technology, 2019). A large part of these losses can be blamed on disease infestation and incorrect storage conditions, eventually leading to rotting or tissue loss. One important crop impacted by bacterial soft rot pathogens (SRP) from the bacterial genera of Dickeya and Pectobacterium includes potatoes. Ten agricultural plants that correspond to 58% of the total global area under cultivation are varieties of potatoes (Velásquez et al., 2018), illustrating the importance of this crop. In the United States alone, potatoes are farmed in 30 states with production corresponding to 4 billion dollars annually (USDA NASS, 2019). There is much to lose from SRP with the expected increase in food demands and impact of climate change upon agriculture.

When considering control mechanisms for SRP, current approaches target pre-planting, planting, growing, harvesting, and grading. In most cases, the attempt is made to limit the conditions that favor the SRP. Traditional activities include utilizing pathogen free seed, application of calcium, avoiding mechanical injury of potato tubers, utilizing clean water for washing, and avoiding residual water on surfaces. These efforts combined lead to reduced infections by these pathogens, however, these methods do not prevent transmission of the pathogen into the field or on to the harvested potatoes. Postharvest application of chlorine dioxide and peroxyacetic acid products can be used to reduce risk of SRP. However, these are aggressive chemicals and are short-acting, reducing their attractiveness for use.

### SUMMARY

Many bacteria communicate using chemical signals, known as quorum sensing (QS), which coordinates the behavior of a bacterial population, e.g., through impacting gene expression related to a variety of traits, under specific conditions. Various human and plant pathogens utilize this chemical communication to cause disease. Some bacteria have the potential to disrupt QS, by producing enzymes that degrade the chemical signals. This process is known as quorum quenching (QQ). QQ may be an alternative way to control infection. As disclosed herein, putative QQ enzymes from seven bacterial genomes were characterized for their ability to degrade acyl-homoserine lactones (AHLs, a QS chemical signal). In particular, bacterial enzymes that could function as QS inhibitors, e.g., against AHL chemical messengers, were produced after identifying genomic targets with probable QQ function and cloning those targets and characterizing putative QQ enzyme expression in E. coli. Enzymes with putative QQ activity were screened against multiple AHLs and against AHL-producing bacteria. Of the 32 cloned enzyme-encoding genes, 24 expressed soluble enzymes. Large scale purification of 14 enzymes with high expression were subsequently examined for activity using seven different pure AHLs and two different bacterial pathogens that produce four AHLs. Results from these various assays illustrated that there were QQ enzymes that caused loss of one or more of the targeted AHLs. Additionally, these enzymes were able to inhibit bacterial biofilm formation for pathogens (e.g., Pectobacterium or other soft-rot pathogens (SRP)). The enzymes are thus likely to have activity against plant pathogens, e.g., virulence or physiology.

In one embodiment, enzymes useful in the compositions and methods include but are not limited to:

P43W_Lactonase5
SEQ ID NO: 1
MTAPFTHGPLRVWSLPTGPIQENAVLIAGEQGQGF
LIDPGDDAGRIAALVAASGVTVTGILLTHAHFDHI
GAVQPLREQLGVPVWLHPDDRELYALGAQSAARWN
LPFTQPAPPDHDITGGQTFTAGDLTLTARHLPGHA
PGHVVFVAPGVVIAGDTLFQGGIGRTDLPGGNHPQ
LLAGIRTQLLTLPDDTAVYPGHGPRTSVGHERRSN
PFL;
P17M_Lactonase3
SEQ ID NO: 2
MSWNHTRQIGQAQVHSLTDGQFRLDGGAMFGSVPK
ALWERAAPADDLNRIRLRINPLLIQLGGENILVET
GFWDQGGEKFEGMYALDRDETVFRGLDRLGLSPED
IHLVINTHLHFDHAGRNVTLLGDPTFPNARYVVQK
QELHDARHTHERSRASYIPAYIDPILDAGLFDVVD
GEHELRPGLSVLPLPGHNLGQQGVVLRSEGQTLVY
VADLIPTLAHAPTPYIMGYDLYPVTTLETRKAHLG
AWFEQNATICTPHDPDAPFARLHENPKGGFTLQAD
S;
P21M_Lactonase2
SEQ ID NO: 3
MSWNHSRQIGQAQVHSLTDGQFRLDGGAMFGSVPR
VLWERAAPADDLNRIRLRINPLLIQLGGENILVET
GFWDQGGEKFEGMYALDRDETVFRGLDRLGLSPED
IHLVINTHLHFDHAGRNVTLLGDPTFPNARYVVQK
QELHDARHTHERSRASYIPAYIDPILDAGLFDVVD
GEHELRPGLSVLPLPGHNLGQQGVVLRSEGQTLVY
VADLIPTLAHAPTPYIMGYDLYPVTTLETRKAHLG
AWFEQNAIICTPHDPDAPFARLHENPKGGFTLQAD
S;
P43W_Lactonase2
SEQ ID NO: 4
MSWNHSRQIGQAQVHSLTDGQFRLDGGAMFGSVPR
VLWERAAPADDLNRIRLRINPLLIQLGGENILVET
GFWDQGGEKFEGMYALDRDETVFRGLDRLGLSPED
IHLVINTHLHFDHAGRNVTLLGDPTFPNARYVVQK
QELHDARHTHERSRASYIPAYIDPILDAGLFDVVD
GEHELRPGLSVLPLPGHNLGQQGVVLRSEGQTLVY
VADLIPTLAHAPTPYIMGYDLYPVTTLETRKAHLG
AWFEQNAIICTPHDPDAPFARLHENPKGGFTLQAD
S;
P17M_Lactonase1
SEQ ID NO: 5
MKRLGDVIVLELPATLMGTPSVIHPVALVGPDHIL
TLVDTGLPGMLDAISGELHAADFTLGQVRRVIVTH
HDLDHIGSLEAVVHATGAEVWALEPEVPYVTGERR
AQKLPSPEQAQAMLADPDLNPTMRAMLTRDPVRVP
VSRALRDGDLLPGQVRVIATPGHTPGHLSLLVPGG
NILISGDALTSQDGALHGPLSRATPDLPGAHDSVR
RLAQEDVQTIVTYHGGVVSDDAGGQLRALA;
P34W_Lactonase2
SEQ ID NO: 6
MKRLGDVIVLELPATLMGTPSVIHPVAPVGPDHIL
TLVDTGLPGMLDAISGELHAADFTLGQVRRVIVTH
HDLDHIGSLEAVVHATGAEVWALEPEVPYVTGERR
AQKLPSPEQAQAMLQEPDLNPVMRALLTREPVRVP
VSRALRDGDLLPGQVRVIATPGHTPGHLSLLVPGG
NILISGDALTSQDGALHGPIPRATPDLPGAHASVR
RLAQEDVQTIVTYHGGVVSDAAGGQLRALA;
P43W_Lactonase4
SEQ ID NO: 7
MKRLGDVIVLELPATLMGTPSVIHPVALVGPDHIL
TLVDTGLPGMLDAIISELHAADFTLGQVRRVIVTH
HDLDHIGSLEAVVHATGAEVWALEPEVPYVTGERR
AQKLPSPEQAQAMLADPDLNPTMRALLTREPVRVP
VSRALRDGDLLPGQVRVIATPGHTPGHLSLLVPGG
NILISGDALTAQGGMLRGPIPRATPDLPGAHDSVR
RLAQEDVQTIVTYHGGVVSDDAGGQLRALAASLD
S;
P21M_Lactonase3
SEQ ID NO: 8
MKRLGDVIVLELPATLMGTPSVIHPVALVGPDHIL
TLVDTGLPGMLDAIIGELHAADFTLGQVRRVIVTH
HDLDHIGSLESVVHATGAEVWALEPEVPYVTGERR
AQKLPSPEQAQAILADPDLNPATRALLTREPTRVP
VSRALRDGDLLPGHVRVIATPGHTPGHLSLLVPGG
NILISGDALTSQDGALHGPLSRATPDLPGAHASVR
RLAQEDVQTIVTYHGGVVSDDAGGQLRALA;
P34W_Lactonase1
SEQ ID NO: 9
VSVRVIPLRAGSCLNLAAITERGAPWRVQAYPAGF
TLILHPTRGPVLFDTGYGADVLTAMRRWPGVIYGL
ITPVQFGPHDSAHEQLRVMGFPPKEVRHIIVSHLH
ADHVGGLRDFPHATFHLDRRAWEPLRALRGVRAVR
RAYLPELLPDDFEDRCTWLDFKEAGNALHPFAEVA
DVFGDGLLRAVPLPGHAPGMVGILAQEDAGLTVLA
ADAAWSVRAGREERPVHPLARVAFHDPAQEATSGA
ALRAFLHANPGARLHVSHDAPEGWT;
P17M_Lactonase2
SEQ ID NO: 10
MSVRVVPLWAGSCLNLSAITERGAPWRVQAYPAGF
TLILHPTRGPVLFDTGYGADVLTAMRRWPGLIYGL
ITPVQFGPHDSAREQLRVLGFPPKEVRHIIVSHLH
ADHVGGLRDFPHATFHLDRRAWEPLRALRGVRAVR
RAYLPELLPDDFEDRCTWLDFKEAGNALHPFAEVA
DVFGDGLLRAVPLPGHAPGMVGILAQEDAGLTVLA
ADAAWSVRAGREERPVHPLARVAFHDPAQEAASGA
ALRAFLHANPGARLHVSHDAPEGWT;
P21M_Lactonase1
SEQ ID NO: 11
VSVRVIPLRAGSCLNLAAITERGAPWRVQAYPAGF
TLILHPTRGPVLFDTGYGADVVTAMRRWPGVIYGL
ITPVQFGPHDSAHEQLRVMGFPPEEVRHIIVSHLH
ADHVGGLRDFPHATFHLDRRAWEPLRALRGVRAVR
RAYLPELLPDDFEDRCTWLDFKEAGNALHPFAEVA
DVFGDGLLRAVPLPGHAPGMVGLLAQEEAGLTVLA
ADAAWSVRAGREERPVHPLARVAFHDPAQEATSGA
ALRAFLHANPAARLHVSHDVPEGWT;
P43W_Lactonase6
SEQ ID NO: 12
MSAQTVTGPVPASKLGFTLPHEHVLFGYPGYQGDL
TLGPFDREAALNACEDVARSLLSRGVRTLVDATPN
ECGRDPAFLRDLSERSGLRILCSSGYYYEGEGAAT
YFKFRASLGGGEAEIEELMRHEVTVGIGSSGVRAG
VIKLASSRDAITPYEQMFFRAAARVQRDTGVPIIT
HTQEGRQGPQQAQLLLSHGADPARIMIGHMDGNTD
PAYHRETLSHGVSVAFDRLGLQGLVGTPTDAQRLD
VLTTLLGEGFADRILLSHDSIWQWLGRPIPMPDAI
LGAVKDWHPLHLTDDILPELERRGVGAEQLRQMTV
GNPARLFG;
P49W_Lactonase1
SEQ ID NO: 13
MMMAAGLHVGARAQGTTTAALTNGAGFYRFKLGDF
TCMVISDGQSTGGNTFPNWGANPGRQEEFGKVLQA
NFIPIEPFTNNFNPMVIDTGKNKVLIDTGRGGTNG
QLLQNLRNAGLTPADIDTVFITHGHGDHIGGMTDA
AGASVFANAKLVMGQQEFDFWASQNNAGFNRNIVP
FKDRFTFVKDGDEIVPGLTAVATPGHTAGHMAVLA
TSGTNKLMHFGDAGGHFLLSLMFPDHYLGFDSNPE
NATATRKKIFEMAANERMMVVGYHYAWPGVGNIRK
KDAAYEFVPTFFRF;
P34W_Lactonase3
SEQ ID NO: 14
MSAQTVTGPVPASELGFTLPHEHVLFGYPGYQGDL
TLGPFDREAALSVCEDVARSLLARGVRTLVDATPN
ECGRDPAFLRDLSERSGLRILCSSGYYYEGEGAAT
YFKFRASLGGGEAEIEELMRHEVTVGIGSSGVRAG
VIKLASSRDAITPYEQMFFRAAARVQRDTGVPIIT
HTQEGRQGPQQAQLLLSHGADPARIMIGHMDGNTD
PAYHRETLSHGVSVAFDRLGLQGLVGTPTDAQRLD
VLTTLLGEGFADRILLSHDSIWQWLGRPIPMPDAI
LGAVKDWHPLHLTDDILPELERRGVGAEQLRQMTV
GNPARLFG;

as well as polypeptides having at least 80%, 82%, 85%, 87%, 89%, 90%, 92%, 94%, 95%, 96%, 97%, 98% or 99% amino acid sequence identity thereto.

Other enzymes which may be useful include but are not limited to those disclosed in Fan et al., Frontiers in Microbio., 11:898 (2020); Zhang et al., Appl. Environ. Microbiol., 85: e02065 (2018); Wang et al., Appl. Environ. Microbiol., 103:21 (2019); Wang et al., Marine Drugs, 17: (2019); Reina et al., Marine Biotech., 21:276 (2019); Barbey et al., Frontiers in Microbio., 9:2800 (2018); See-Too et al., Microbial Cell Factories, 17:179 (2018); Shastry et al., FEMS Micro. Lett., 365:fny54 (2018); Torres et al., Sci. Rep., 7:943 (2017); Hosseinzadeh et al., Arch. Microbio., 199:51 (2017); Gomez-Garzon et al., Can. J. Microbiol., 63:74 (2017); Garge et al., PLoS One, 11: e0167344 (2016), which are incorporated by reference herein

In one embodiment, a method to prevent, inhibit or treat soft rot in a vegetable, fruit or ornamental plant, e.g., cabbage, is provided. The method includes contacting the vegetable, the fruit or the ornamental crop plant with a composition comprising an effective amount of one or more quorum quenching enzymes, e.g., bacterial lactonases. In one embodiment, the vegetable is a potato. In one embodiment, the vegetable is a cucumber. In one embodiment, the lactonase is a metal dependent hydrolase. In one embodiment, the lactonase is a metallo-beta-lactamase. In one embodiment, the composition is applied to, e.g., via spraying or rinsing, the vegetable, fruit or ornamental crop plant. In one embodiment, the vegetable is suspected of being infected with a pathogen. In one embodiment, the pathogen comprises Dickeya or Pectobacterium. In one embodiment, the pathogen comprises Erwinia, Pectobacterium or Pseudomonas. In one embodiment, the lactonase is from Deinococcus. In one embodiment, the lactonase has at least 80% amino acid sequence identity to one of SEQ ID Nos. 1-39. In one embodiment, the lactonase comprises a protein that opens the lactone ring on an acyl homoserine lactone molecule. In one embodiment, the enzyme is from Ochrobactrum sp., e.g., intermedium D-2. In one embodiment, the enzyme is from Bosea sp., e.g., AHL lactonase, AidB. In one embodiment, the enzyme comprises momL, e.g., MomL(I144V), MomL (L254R), and/or MomL(V149A). In one embodiment, the enzyme is from Stenotrophomonas sp., e.g., S. maltophilia. In one embodiment, the enzyme is from Rhodococcus sp., e.g., R. erythropolis. In one embodiment, the enzyme comprises lactonase QsdA. In one embodiment, the enzyme is from Planococcus sp., e.g., P. versutus. In one embodiment, the enzyme comprises AidF or AidP. In one embodiment, the enzyme is from Enterobacter sp or Kurthia sp., e.g., Khuakui LAM0618 T. In one embodiment, the enzyme comprises AiiE or AiiK. In one embodiment, the enzyme comprises AiiA. In one embodiment, the enzyme is from Bacillus sp., e.g., B. thuringiensis. In one embodiment, the enzyme comprises HqiA. In one embodiment, the enzyme is from Lysinibacillus sp., L. sphaericus. In one embodiment, the enzyme is from Geobacillus sp. In one embodiment, the enzyme comprises AdeH. Exemplary plants are disclosed in Table 1 of Charkowski, Ann. Rev., 56:269 (2018), which is incorporated by reference herein.

In one embodiment, a method to decrease virulence or inhibit a quorum sensing pathogen is provided. The method includes applying a composition comprising an effective amount of one or more quorum quenching enzymes, e.g., bacterial lactonases. In one embodiment, the lactonase is a metal dependent hydrolase. In one embodiment, the lactonase is a metallo-beta-lactamase. In one embodiment, the lactonase is a phosphotriesterase like protein. In one embodiment, the lactonase is a Zn-dependent hydrolase. In one embodiment, the composition is applied via spraying or rinsing. In one embodiment, the pathogen comprises Dickeya or Pectobacterium. In one embodiment, the pathogen comprises Erwinia, Pectobacterium or Pseudomonas. In one embodiment, the lactonase is from Deinococcus species. In one embodiment, the enzyme has at least 80% amino acid sequence identity to one of SEQ ID Nos. 1-39.

In one embodiment, the enzymes may be employed to prevent, inhibit or treat soft rot in crops including but not limited to potatoes, cucumber, carrots, Chinese cabbage, peppers, onions, zucchini, or celery, e.g., as a result of infection or risk of infection with Pectobacteria species, for example, Pectobacteria atrosepticum or Pectobacteria carotovorum.

In one embodiment, a method to produce lactonases is provided. The method includes expressing in a host cell, e.g., a bacteria, yeast or insect cell, a vector comprising an expression cassette comprising a heterologous promoter operably linked to an open reading frame encoding a lactonase; and isolating the expressed lactonase. In one embodiment, the lactonase is not secreted into a culture medium. In one embodiment, the lactonase is from Deinococcus. In one embodiment, the lactonase has at least 80% amino acid sequence identity to one of SEQ ID Nos. 1-39. Isolated lactonase prepared by the method is also provided.

In one embodiment, an isolated host cell or a nucleic acid vector comprising a heterologous promoter operably linked to an open reading frame encoding a bacterial lactonase is provided, i.e., the host cell or nucleic acid is recombinant. In one embodiment, the host cell is a bacterial host cell. In one embodiment, the host cell is a plant cell, e.g., in a transgenic plant, that expresses one or SEQ ID Nos. 1-39 or a lactonase enzyme with at least 80%, 82%, 85%, 87%, 90%, 92%, 94%, 95%, 97%, 98%, or 99% amino acid sequence identity to one of SEQ ID Nos. 1-39. In one embodiment, the transgenic plant is a vegetable plant. In one embodiment, the transgenic plant is a fruit plant. In one embodiment, the transgenic plant is an ornamental plant. In one embodiment, the host cell is a bacterial host cell.

In one embodiment, a composition comprising one or more isolated bacterial lactonases, e.g., one or more soluble lactonases, and optionally a carrier, is provided. In one embodiment, the carrier comprises a pH-buffered water solution, a pH-buffered saline solution, a surfactant-containing water solution, a metal salt-containing water solution or any other solution or powder that does not denature the enzyme.

The present compositions are unlike peroxyacetic acid and chlorine dioxide treatments, which are aggressive chemicals and are nonspecific. Moreover, the present composition may be employed with other anti-microbials, e.g., Bio-Save which is a formulation containing a microorganism that controls mold-based diseases dry rot and silver scurf. In one embodiment, the present compositions may be applied simultaneously or sequentially with other products.

### BRIEF DESCRIPTION OF THE FIGURES

FIGS. 1A-1C. Amino acid sequences of A) Pseudomonas aeruginosa acylases (PvdQ; SEQ ID Nos:15-18) and), B) Bacillus thuringiensis hydrolases (AiiA; SEQ ID Nos. 19-21) and C) Rhodococcus hydrolases (QsdA; SEQ ID Nos.22-24).

FIG. 2. AHL (quorum sensing) signaling.

FIG. 3. Quorum quenching.

FIG. 4. Pathogen control using QQ.

FIG. 5. Exemplary system for QQ intervention.

FIG. 6. Exemplary isolates having AHL degrading enzymes.

FIG. 7. Summary of identification method.

FIG. 8. A) Three AHLs examined for degradation, from top to bottom: 3oxo-C6-HSL, C6-HSL, 3oxo-C8-HSL, and B) Conversion of 3oxo-C6-HSL by lactonase enzyme.

FIG. 9. Impact of Deinococcus species putative lactonase enzymes upon Pectobacterium carotovorum (Pcc) and Pectobacterium atrosepticum (Pca) growth in pure culture. (A.) growth in tryptic soy broth after 24 hours and (B.) growth with respect to control. The vertical lines indicate related putative lactonase enzymes. One-way ANOVA were statistically significant (p-value 0.000). * indicate means that were statistically different from untreated (control).

FIG. 10. Impact of Deinococcus species putative lactonase enzymes upon Pectobacterium carotovorum (Pcc) and Pectobacterium atrosepticum (Pca) upon biofilm formation in pure culture. (A.) biofilm formation in tryptic soy broth and (B.) biofilm formation with respect to control. The vertical lines indicate related putative lactonase enzymes. One-way ANOVA were statistically significant (p-value 0.000). * indicate means that were statistically different from untreated (control).

FIG. 11. Impact of Deinococcus species putative lactonase enzymes upon Pectobacterium carotovorum (Pcc) in potato tissue after a 4-hour exposure. (A.) cell counts as colony forming units, and (B.) cell counts as percent control. The corresponding LC-MS analyses illustrated that six putative lactonase enzymes results in loss of three different AHL (C6, C8, and oxo-C8) leading to near-blank peak areas or non-detects in the chemical analysis.

FIG. 12. Impact of Deinococcus species putative lactonase enzymes upon Pectobacterium atrosepticum (Pca) in potato tissue after 4-hour exposure. (A.) cell counts as colony forming units, and (B.) cell counts as percent control. The corresponding LC-MS analyses illustrated that six putative lactonase enzymes results in loss of three different AHL (C6, C8, and oxo-C8) leading to near-blank peak areas or non-detects in the chemical analysis.

FIG. 13. Alignments for enzymes useful in the compositions and methods (SEQ ID Nos. 25-39).

FIG. 14. Impact of Deinococcus species putative lactonase enzymes upon Pectobacterium carotovorum (Pcc) (orange) and Pectobacterium atrosepticum (Pca) (blue) growth in pure culture. (A.) growth in tryptic soy broth after 24 hours and (B.) growth with respect to control. The vertical lines indicate related putative lactonase enzymes. One-way ANOVA were statistically significant (p-value 0.000). * indicate means that were statistically different from untreated (control).

FIG. 15. Impact of Deinococcus species putative lactonase enzymes upon Pectobacterium carotovorum (Pcc) (orange) and Pectobacterium atrosepticum (Pca) (blue) upon biofilm formation in pure culture. (A.) biofilm formation in tryptic soy broth and (B.) biofilm formation with respect to control. The vertical lines indicate related putative lactonase enzymes. One-way ANOVA were statistically significant (p-value 0.000). * indicate means that were statistically different from untreated (control).

FIG. 16. Impact of Deinococcus species putative lactonase enzymes upon Pectobacterium carotovorum (Pcc) in potato tissue after a 4-hour exposure. (A.) cell counts as colony forming units and (B.) cell counts as percent control. The corresponding LC-MS analyses illustrated that six putative lactonase enzymes results in loss of three different AHL (C6, C8, and oxo-C8) leading to near-blank peak areas or non-detects in the chemical analysis.

FIG. 17. Impact of Deinococcus species putative lactonase enzymes upon Pectobacterium atrosepticum (Pca) in potato tissue after 4-hour exposure. (A.) cell counts as colony forming units and (B.) cell counts as percent control. The corresponding LC-MS analyses illustrated that six putative lactonase enzymes results in loss of three different AHL (C6, C8, and oxo-C8) leading to near-blank peak areas or non-detects in the chemical analysis.

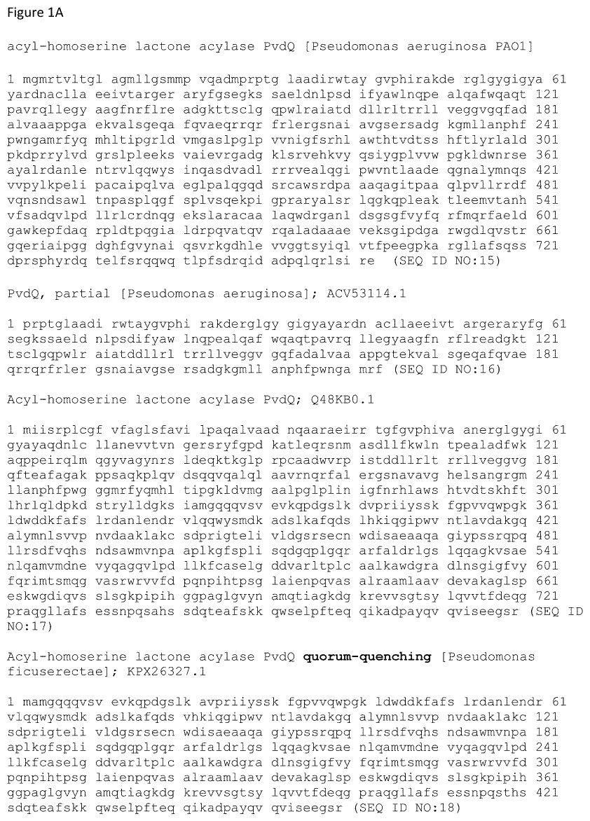

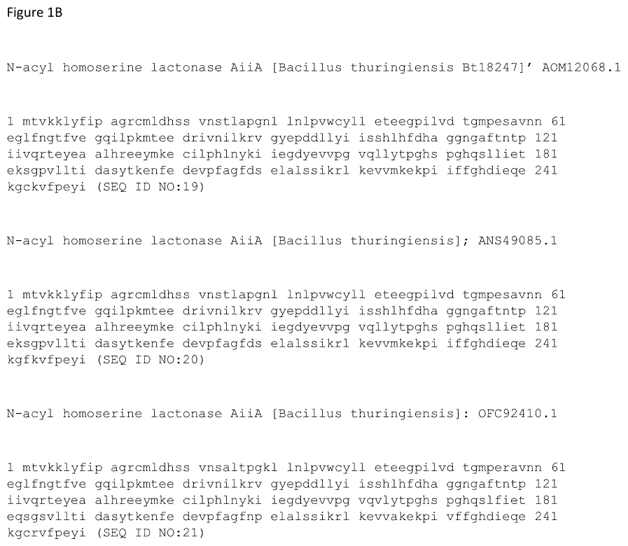

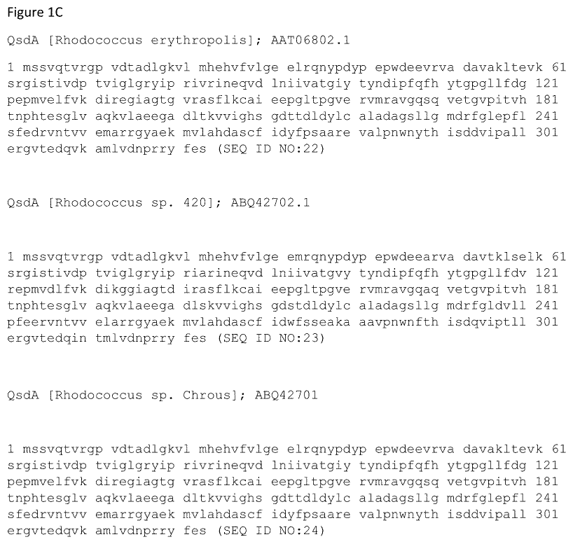

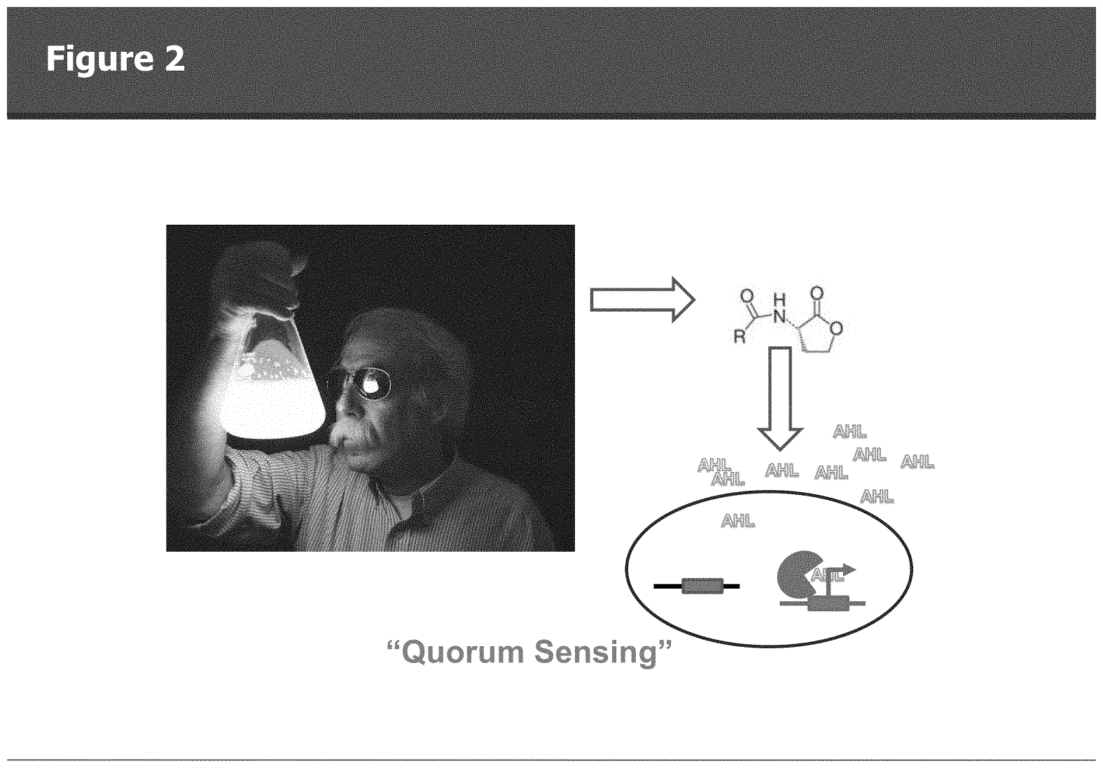

### DETAILED DESCRIPTION

### Definitions

A “vector” refers to a macromolecule or association of macromolecules that comprises or associates with a polynucleotide, and which can be used to mediate delivery of the polynucleotide to a cell, either in vitro or in vivo. Illustrative vectors include, for example, plasmids, microbial vectors such as bacterial or viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest), a coding sequence of interest in vaccine development (such as a polynucleotide expressing a protein, polypeptide or peptide suitable for eliciting an immune response in a mammal), and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by heterologousization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

As used herein, the terms “isolated and/or purified” refer to in vitro preparation, isolation and/or purification of a microbial strain, cell or protein, so that it is not associated with and/or is substantially purified from in vitro or in vivo substances. An isolated strain or cell preparation of the invention is generally obtained by in vitro culture and propagation. A “recombinant” protein is one expressed using recombinant DNA techniques and a “recombinant” strain or cell is one which has been manipulated in vitro, e.g., using recombinant DNA techniques to introduce changes to the host genome. For example, a “recombinant” strain or cell of the invention may be one which has been manipulated in vitro so as to contain an insertion and/or deletion of DNA in the genome, e.g., chromosome, of the strain or cell relative to the genome, e.g., chromosome, of the parent strain or cell from which the recombinant strain or cell was obtained (e.g., “wild-type” strain). In one embodiment, an insertion in the recombinant strain is stable, e.g., the insertion and its corresponding phenotype do not revert to wild-type after numerous passages. Included within the scope of the phrase “recombinant strain” is one which, through homologous recombination, includes a gene which contains a mutation that results in the inactivation of the protein in or reduced expression of the gene, e.g., results in a polypeptide having reduced or lacking biological activity or so that the polypeptide is not expressed, relative to a corresponding wild-type strain that does not include the recombined gene.

Thus, an “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. Increasing enrichments of the embodiments of this invention are increasingly preferred. Thus, for example, a 2-fold enrichment, 10-fold enrichment, 100-fold enrichment, or a 1000-fold enrichment.

A “transcriptional regulatory sequence” refers to a genomic region that controls the transcription of a gene or coding sequence to which it is operably linked. Transcriptional regulatory sequences of use in the present invention generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present invention are provided below.

“Host cells,” “cell lines,” “cell cultures,” “packaging cell line” and other such terms denote higher eukaryotic cells, such as mammalian cells including human cells, useful in the present invention, e.g., to produce recombinant virus or recombinant fusion polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphonylation, lipidation, or conjugation with a labeling component.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells of the present invention are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 85%, 90%, 95% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

“Conservative” amino acid substitutions are, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic; trp, tyr, phe.

For example, in one embodiment, a protein or recombinant nucleic acid molecule encoding the protein for use in the compositions and methods of the invention has up to 5% of the residues, e.g., 1, 2, 3, or 4 residues substituted, up to 10% of the residues substituted, e.g., with conservative substitutions, or up to 20% of the residues in substituted, or any combination thereof, relative to any one of SEQ ID Nos. 1-39. In one embodiment, a protein for use in the compositions and methods of the invention has up to 5% of the residues substituted with conservative substitutions, up to 5%, e.g., 1 or 2, of the residues substituted with conservative substitutions, or up to 20% of the residues substituted, or any combination thereof, relative to SEQ ID Nos. 1-39. Whether a particular amino acid substitution results in a functional polypeptide can readily be determined by assaying the biological activity of the variant polypeptide by methods well known to the art. For example, a protein may have at least 80%, 85%, 90%, 92%, 95%, 97%, 98%, or 99% amino acid identity over the complete sequence of SEQ ID Nos. 1-39, and the substituted residues may be conservative or non-conservative substitutions.

For example, in one embodiment, a protein for use in the compositions and methods of the invention has up to 5% of the residues in one of SEQ ID Nos. 1-39 substituted with conservative or nonconservative substitutions, up to 10% of the residues in one of SEQ ID Nos. 1-39 substituted with conservative or nonconservative substitutions, or up to 20% of the residues in one of SEQ ID Nos. 1-39 substituted, or any combination thereof. In one embodiment, a protein for use in the compositions and methods of the invention has up to 5% to 10% of the residues in one of SEQ ID Nos. 1-39 substituted with conservative substitutions, up to 15% of the residues in one of SEQ ID Nos. 1-39 substituted with conservative substitutions, or up to 20% of the residues one of SEQ ID Nos. 1-39 substituted, or any combination thereof. Whether a particular amino acid substitution results in a functional polypeptide can readily be determined by assaying the biological activity of the variant polypeptide by methods well known to the art.

In one embodiment, a protein for use in the compositions and methods of the invention has 1, 2, 3, 4, 5, 6, 7, 8, 9, 10 or 11 residues, in SEQ ID Nos. 1-39 substituted with conservative or nonconservative substitutions. Whether a particular amino acid substitution results in a functional polypeptide can readily be determined by assaying the biological activity of the variant polypeptide by methods well known to the art.

The invention also envisions polypeptides with non-conservative substitutions. Non-conservative substitutions entail exchanging a member of one of the classes described above for another.

In one embodiment, a composition of the invention comprises one or more isolated enzyme(s), or recombinant virus or host cells expressing one or more of the enzymes in an amount effective to elicit an antimicrobial response. For instance, recombinant protein may be isolated from a suitable expression system, such as bacteria, insect cells or yeast, e.g., E. coli, L. lactis, Pichia or S. cerevisiae or other bacterial, insect or yeast expression systems, or mammalian expression systems such as T-REx™ (Invitrogen). For example, to prepare isolated recombinant enzymes, any suitable host cell may be employed, e.g., E. coli or yeast, to express those proteins. Those cellular expression systems may also be employed as delivery systems, e.g., E. coli, expressing a heterologous lactonase, such as one expressed on the cell surface or in a secreted form. Thus, the recombinant enzyme useful in the compositions and methods of the invention may be expressed in, or on the surface of, a prokaryotic or eukaryotic cell, or may be secreted by that cell, and may be expressed as a fusion, e.g., a His tag may be fused to the recombinant protein, or the recombinant protein may be fused to a molecule with a distinct function, e.g., linked to a molecule that alters solubility (e.g., prevents aggregation) or half-life, e.g., a PEGylated molecule, of the resulting molecule.

In one embodiment, the invention provides a method of treating, inhibiting or preventing a bacterial infection, e.g., of a vegetable, fruit or ornamental plant. In one embodiment, the method comprises administering, e.g., applying, an effective amount of a composition of the invention before, during or after exposure to the bacterium. In one embodiment, the composition comprises isolated enzyme, either native or recombinant, from one or more sources. In one embodiment, the method comprises administering an effective amount of a composition of the invention after exposure to the bacterium.

As will be apparent to one skilled in the art, the optimal concentration of the active agent in a composition of the invention will necessarily depend upon the specific agent(s) used, the characteristics of the product to which it is applied, the type and amount of carrier or other bioactive agent, if any, and the nature of the microbial infection. These factors can be determined by those of skill in the relevant arts in view of the present disclosure. In general, the active agent(s) in the composition of the invention are administered at a concentration that either modulates antimicrobial activity or modulates quorum sensing molecules, e.g., inhibits the production or amount of those molecules.

Specific dosages may be adjusted depending on conditions of the environment, e.g., temperature, moisture, light exposure and the like, or type, age, and/or weight of vegetable, fruit or ornamental plant, and application method. Any of the dosage forms described herein containing effective amounts are well within the bounds of routine experimentation and therefore, well within the scope of the instant disclosure.

A composition may comprise an enzyme described herein in an amount of from about 100 μg per mL to about 1000 μg per mL, in some instances from about 200 μg per mL to about 1000 μg per mL, and in some instances from about 500 μg per mL to about 1000 μg per mL. In one embodiment, the composition may comprise an enzyme described herein in an amount of from about 1 μM to about 1000 μM, in some instances from about 10 μM to about 100 μM, about 100 μM to about 200 μM, about 200 μM to about 300 μM, about 300 μM to about 400 μM , about 400 μM to about 500 μM, about 500 μM to about 600 μM, about 600 μM to about 700 μM and in some instances from about 800 μM to about 1000 μM. In one embodiment, the composition comprises an amount of about 1 μg to about 200 μg of enzyme per dose for a target, e.g., a plurality of potatoes, weighing about 20 to 25 μg. In one embodiment, the composition comprises an enzyme described herein in an amount of about 1 mg to about 1000 mg, e.g., about 10 mg to about 100 mg, or an amount of about 0.1 μg to about 1000 μg, e.g., about 1 μg to about 10 μg . In one embodiment, the composition comprises an enzyme in an amount of about 20 μg/kg to about 2000 μg/kg, e.g., about 50 μg/kg to about 500 μg/kg or about 100 μg/kg to about 400 μg g/kg.

The desired amount of the composition may be applied over time or in one application. Optionally, a dose of composition may be administered on one day, followed by one or more other doses spaced as desired thereinafter. In one exemplary embodiment, an initial dose is given, followed by a boost of the same composition approximately two to four days later. In one particular embodiment, a first dose of the composition is administered followed by a second dose at about 24 hours to about 96 hours after the first dose. Other dosage schedules may also be used.

In addition to the isolated enzyme(s), recombinant virus, or recombinant cells, or combinations thereof, the composition of the invention may further comprise one or more suitable carriers. As used herein, the term “acceptable carrier” refers to an acceptable vehicle for administering a composition comprising one or more non-toxic excipients which do not react with or reduce the effectiveness of the active agents contained therein. The proportion and type of acceptable carrier in the composition may vary, depending on the chosen route of application.

Optionally, the composition may further comprise minor amounts of auxiliary substances such as agents that enhance the antimicrobial effectiveness of the preparation, stabilizers, preservatives, and the like.

In one embodiment, the composition may also comprise a bile acid or a derivative thereof, in particular in the form of a salt. These include derivatives of cholic acid and salts thereof, in particular sodium salts of cholic acid or cholic acid derivatives. Examples of bile acids and derivatives thereof include cholic acid, deoxycholic acid, chenodeoxycholic acid, lithocholic acid, ursodeoxycholic acid, hydroxycholic acid and derivatives such as glyco-, tauro-, amidopropyl-1-propanesulfonic-, amidopropyl-2-hydroxy-l-propanesulfonic derivatives of the aforementioned bile acids, or N,N-bis (3Dgluconoamidopropyl) deoxycholamide. A particular example is sodium deoxycholate (NaDOC).

Examples of suitable stabilizers include protease inhibitors, sugars such as sucrose and glycerol, encapsulating polymers, chelating agents such as ethylene-diaminetetracetic acid (EDTA), proteins and polypeptides such as gelatin and polyglycine and combinations thereof.

Depending on the route of application, the compositions may take the form of a solution, suspension, emulsion, or the like.

### Exemplary Compositions and Methods

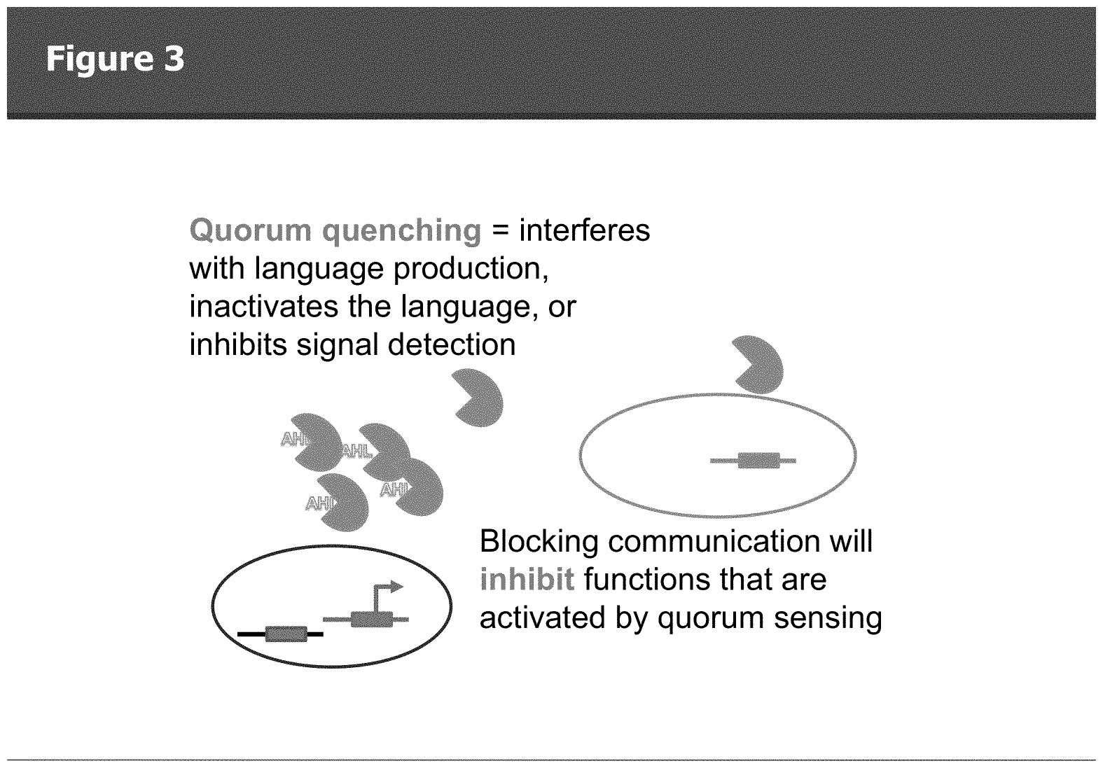

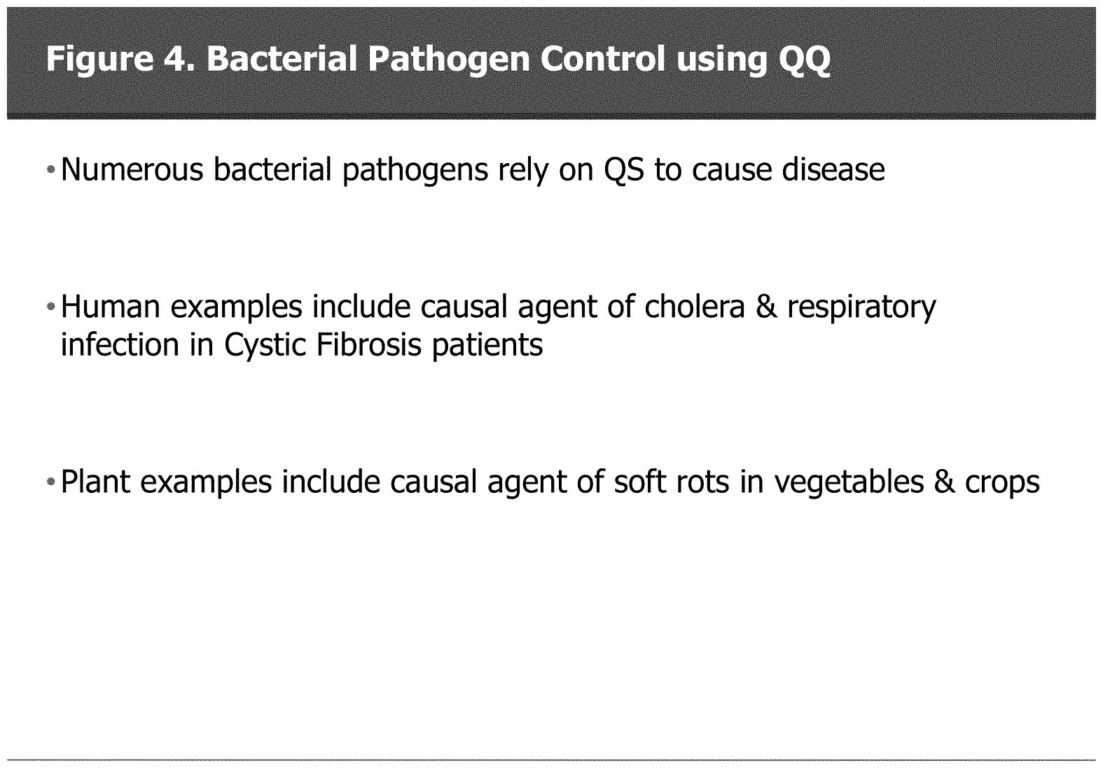

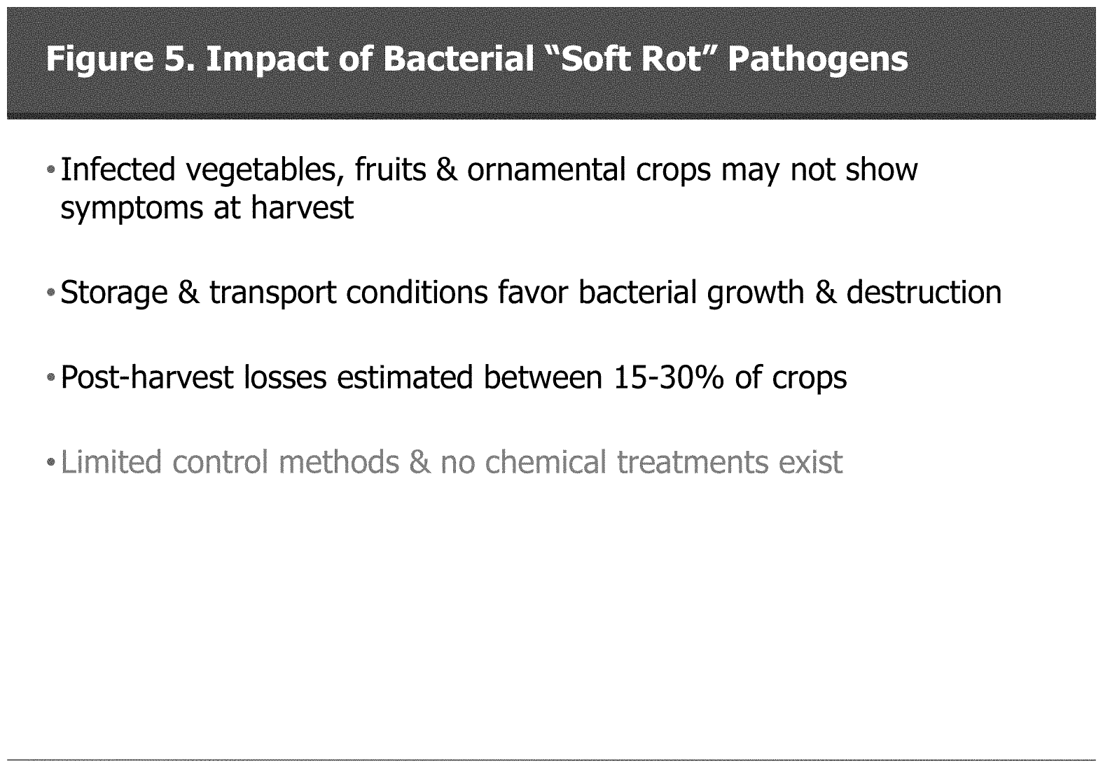

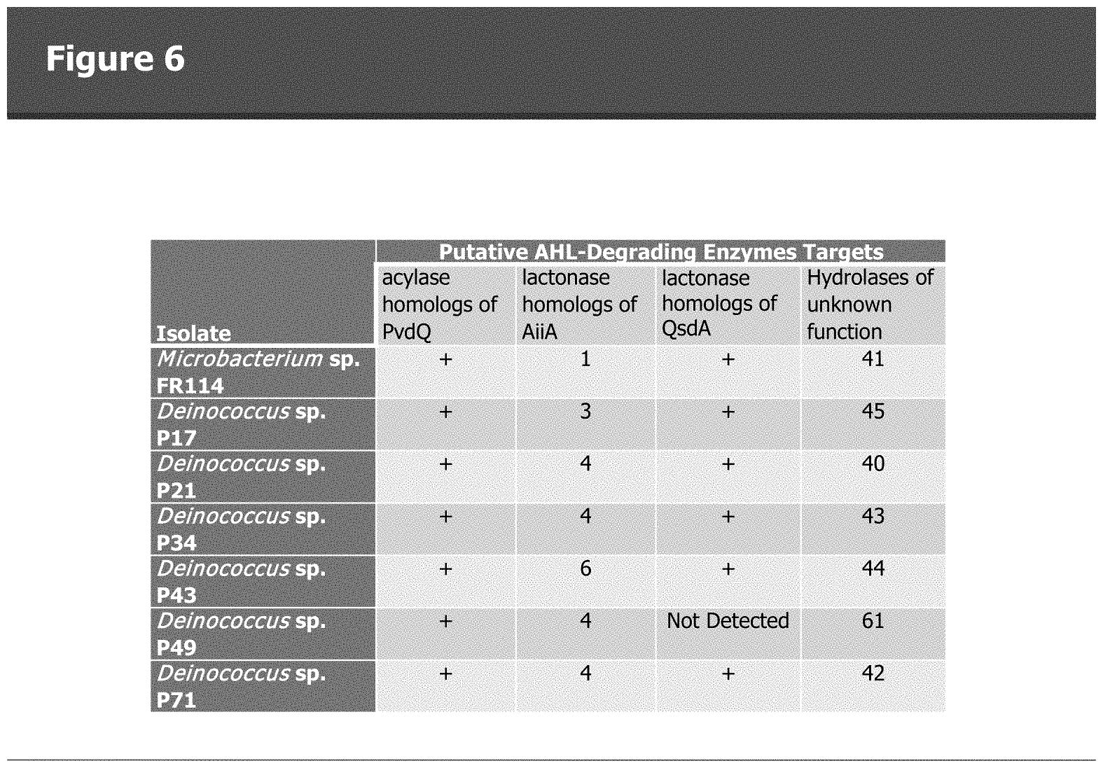

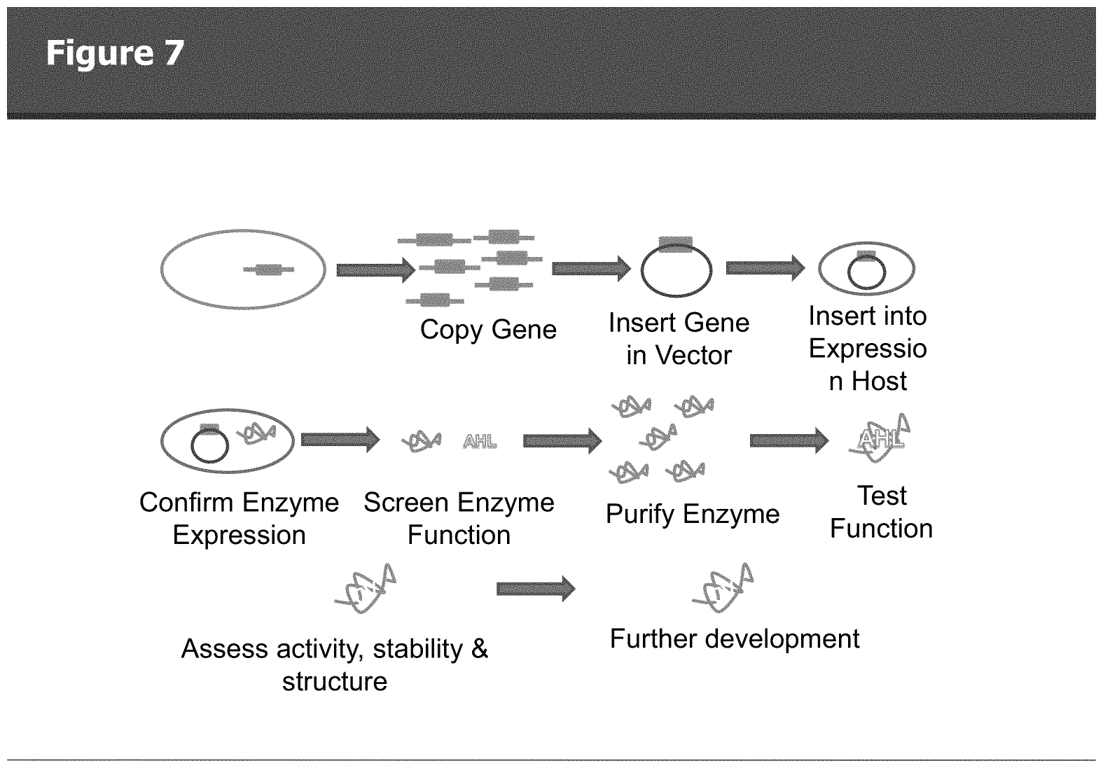

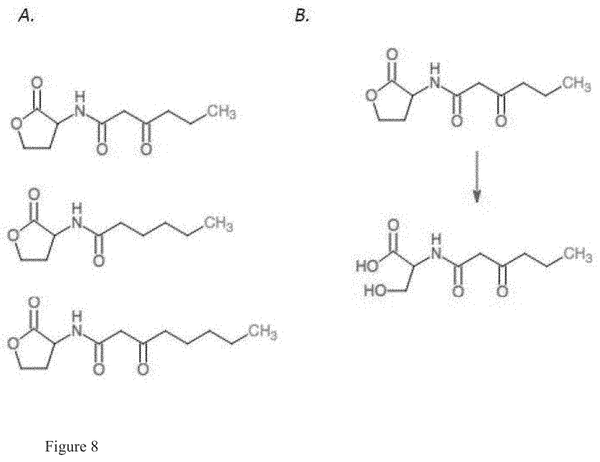

One important aspect of SRP is the need for bacteria to communicate with each other, leading to rapid colonization and activity against the plant tissue. As disclosed herein, a biocontrol agent composed of one or more functional enzymes that target and degrade bacterial communication molecules is provided. Many bacteria use N-acyl homoserine lactones (AHLs) for communication (FIG. 2). Degradation of these chemical signals would reduce the effects of SRP pathogens. Enzymatic options for plants, more specifically vegetable protection, would allow growers and suppliers a non-harmful way to limit economic losses. An enzymatic biocontrol agent would not exhibit the toxicity of currently-used chemicals, but it could be co-applied with treatments for other diseases and would be easily re-applied to plants during a growing season. In one embodiment, an enzyme containing composition may be directly applied to a crop such as potatoes or cucumbers. Effective control of SRP would improve yields and increase the percentage of harvested crops that reach consumers.

Numerous bacterial species belonging to the phylum Proteobacteria communicate with each other using acyl-homoserine lactones (AHL), a chemical language. Sensing these AHL molecules results in the bacterial species turning on various traits, e.g., gene regulation, known as quorum sensing (QS). The concern surrounding QS is the number of pathogens, both plant and animal, that turn on various virulence genes when sensing their chemical language. Pectobacterium carotovorum is a plant pathogen that exhibits QS and increases its virulence.

In order to provide alternative ways to control plant pathogens, the quorum sensing potential of bacteria, e.g., lactonases from Deinococcus isolates, was employed. Lactonases have the ability to open the AHL-lactone ring thereby rendering this signaling molecule inactive. This process is known as quorum quenching (QQ). Deinococcus genes for these molecules were cloned into E. coli, the bacteria cultured to overexpress the enzymes and the enzymes isolated. Using both analytical techniques (LC-MS) and biological tests (biofilm assays and plant tissue infection), it was determined that these lactonases are QQ proteins.

Specifically, thirty-two putative Deinococcus spp. QQ enzymes were successfully cloned and expressed in an E. coli host. Twenty-four of 32 were soluble (e.g., well behaved) allowing for further study. Large scale purification of 14 enzymes with the highest expression were subsequently examined for activity against seven different pure AHLs and four different bacterial pathogens that produce four AHLs. These various assays illustrated QQ enzymes that cause loss of the AHL. Enzymes were able to inhibit bacterial biofilm formation for two opportunistic human bacterial pathogens (e.g., S. marcesens and P. aeruginosa). AHL loss and decreased bacterial biofilm formation may be employed to determine the efficacy of the enzymes. Direct application experiments and measurement of, e.g., expression and enzyme activity, determine if the enzymes (n=14) work against a target plant pathogen, Pectobacterium carotovorum and Pectobacterium atrosepticum, limiting its ability to cause soft rot.

Thus, these QQ enzymes are an alternative to integrated pest management (IPM) techniques that focus on things such as crop rotation, soil/field conditions, and seed quality. These IPMs may successfully reduce all loss as the target pathogen is a common soil bacterium. Thus, the enzymes, e.g., delivered via a vector or delivery vehicle, may reduce crop loss post-harvest and serve as a seed treatment prior to planting. One advantage of protein application is that you do not need a GMO plant that produces these enzymes, but instead can utilize techniques that serve as a topical application.

The enzyme(s) or host cells that exogenously express the enzyme(s) may be used alone or to supplement current methods to reduce soft rot, e.g., for potatoes by lowering the temperature, providing airflow and reducing humidity and for cucumbers by using insecticides to control the cucumber beetles. In one embodiment, the isolated enzyme(s) or host cells expressing the enzyme(s) are applied to vegetable or fruit, e.g., post-harvest including during storage, processing or prior to stocking. In one embodiment, the isolated enzyme(s) or host cells expressing the enzyme(s) are applied to vegetable plant, fruit plants or ornamental crop plants. In one embodiment, the isolated enzyme(s) may be used to reduce the incidence of soft rot in stored potatoes and bacterial wilt in cucumbers, which are caused by bacterial infection by species naturally present in the environment that rely on quorum sensing as they become pathogenic.

The invention will be further described by the following non-limiting examples:

### EXAMPLE 1

### Summary

As described below, the bacterial enzymes that were produced are effective at degrading the bacterial communication molecules in vivo. Several of the enzymes act as lactonases (ring-opening) for homoserine lactones (HSL—common bacterial communication molecules) in vitro. Cultures of SRP bacteria are cultured with the enzymes HSLs, e.g., intact HSLs. Potato pieces are infected with the SRP bacteria and the enzymes to determine if the SRP process is interrupted or retarded, e.g., based on gene expression, e.g., detection of AHL, and growth.

### Introduction

Efforts to reduce the effects of SRP has not greatly changed in the last 20 years. These strategies can be effective but rely on the ability of another bacterium to flourish in a specific environment. Most current efforts focus on “creating” a more favorable environment for plant growth, whereas the present technology addresses a need in the post-harvest arena.

SRPs are generically classified by their ability to macerate fruit, vegetable, and plant tissues, reducing these economically and globally valuable products to waste either in the field, storage, or transport (Põllumaa et al., 2012; Charkowski et al., 2018). Numerous agriculturally important crops (e.g., potatoes, cucumbers, cabbage, tomatoes, and the like) can be the victims of these bacterial SRP (also known as soft-rot Enterobacteriaceae), corresponding to the bacterial genera of Dickeya and Pectobacterium (Charkowski et al., 2018). The mechanisms used to digest the plant tissue are orchestrated by a gene regulation mechanism known as quorum sensing (QS) (Barnard and Salmond, 2007). In the case of Pectobacterium strains, they synthesize N-acyl-homoserine lactones, dominantly 3-oxo-C6-HSL, and 3-oxo-C8-HSL (Crepin et al., 2012) (FIG. 8A). These small chemical signaling molecules are synthesized by the bacterium under cell density control, thus when cell densities are large enough, receiving these chemical signals leads to changes in gene expression and subsequently function (Lee et al., 2013). In the case of Pectobacterium strains, binding of the 3-oxo-C6 or C8 HSLs to ExpR/EsrA leads to removing repression on the plant cell wall degrading enzymes (PCWDE), thereby allowing the bacterium to synthesize enzymes (e.g., cellulases, proteases, or pectinases) required for virulence (Barnard and Salmond 2007). A viable option for limiting the effects of SRP revolves around the antagonist approach of quorum quenching (QQ) to degrade the chemical signals. If the extracellular levels of AHLs are reduced, then behaviors regulated by detection of these chemical signals would likewise be reduced.

Experimental

In one embodiment, QQ enzymes from bacteria belonging to the genera of Deinococcus were investigated. A genomic and proteomic approach yielded a handful (n=8) of functional lactonase enzymes capable of opening the lactone ring, effectively quenching the AHL signal (FIG. 8B). Under in vitro chemical assays the six enzyme groups reduced levels of 3-oxo-C6-HSL and 3-oxo-C8-HSL. In addition, when the enzymes were tested against two AHL-producing bacterial pathogens, the physiological response of biofilm formation was reduced. These combined outcomes indicated active lactonases with the ability to impact QS regulated function were produced. To assess these putative lactonases as a plant protective product against the SRP organism, Pectobacterium carotovora and Pectobacterium atrosepticum, model culture conditions are used to determine which enzymes work most efficiently upon pathogen growth and QS phenotypes (e.g., biofilm formation). Plant degradation and gene expression assays are also conducted to determine how virulence is attenuated. Attenuation of the AHL levels when the culture is exposed to the lactonases which in turn results in reduced virulence by controlling PCWDE with less tissue damage in the vegetable.

Lactonases are enzymes that catalyze the opening of the lactone ring structure (FIG. 8B). 14 putative lactonase enzymes from different strains of Deinococcus were cloned and isolated. These predicted enzyme sequences corresponded to metal or zinc dependent hydrolyses, metallo-beta-lactamases, phosphotriesterase like proteins, hydroyacylglutathione hydrolases, or Zn-dependent hydrolases based upon protein similarity. When these enzymes were challenged with different AHLs, several showed evidence of lactonase activity against the AHLs synthesized by a pathogenic target Pectobacterium species Pcc and Pca The enzymes function in the same manner when challenged against the bacterium directly, showing enzyme specificity for the target small molecules and ability to function under non-optimal conditions, and allowing for determining the rate of degradation. These three outcomes allow ranking of enzyme activity. The bacterial pathogen, P. carotovora (Pcc) and P. atrosepticum (Pca) were grown in a polygalacturonic acid (PGA) mineral salt medium (Crepin et al., 2012). The PGA media is used because it is the main structure of the plant polymer pectin and serves as an effective carbon source for the organism. Cultures are grown into early exponential phase, then inoculated into fresh PGA mineral salt media, ensuring that the expI gene has been expressed to enhance the rate of AHL production.

Pcc and Pca regulates numerous aspects of its physiology and specifically pathogenesis towards plants occurs through a complex QS regulatory network (Barnard and Salmond, 2007; Põllumaa et al 2012). Production of PCWDE (e.g., polygalacturonase, pectate lyase, cellulase, xylanase, and protease) revolves around the QS circuit (Charkowski et al 2018). This circuit includes production of AHL by ExpI/EsaI and signal sensing by ExpR/EsaR, which controls expression of regulatory protein RsmA. RsmA serves as a gate keeper to silence the PCWDE, thereby when AHLs are not detected, PCWDE are kept silenced (Barnard and Salmond, 2007; Põllumaa et al 2012; Valente et al 2017). But when AHLs are detected, this limits repression by RsmA thereby expression of various virulence functions occurs. To characterize the effect of the lactonases on the physiology of Pcc and Pca, a tiered approach is used to address both gene regulation and the effects of various virulence traits indirectly and directly on potato tubers. In order to understand gene expression, quantitative-PCR assays for the QS circuit, regulator rsmA, and three PCWDE are conducted for which gene expression are compared to housekeeping genes to normalize expression. These qPCR assays are tested in PGA minimal media and standard medium (tryptic soy broth) to assess sensitivity. Time series and infectious dose pathogen progression assays are conducted to confirm gene expression of QS circuit, regulator rsmA, and PCWDE. These pathogenicity assays are according to the methods in Dong et al. (2004). Briefly, Pcc and Pca is grown in a PGA minimal salt media. Potato pieces are surface sterilized to ensure removal of native bacteria and Pcc or Pca inoculums are injected on the potato piece (Garge and Nerurkar, 2017). The infection is followed for 4 hr initially and then again in Pcc at 10-12 and 24-hour intervals. Each time point is run with three biological replicates to clearly determine gene expression, cell growth and AHL levels. Tissue maceration was tested at 24 hours post infection. Tissue maceration is determined by characterizing percent of area damaged and change in weight after removing the macerated region to calculated percentage of tissue loss to infection (Garge and Nerurkar, 2017). Tissue samples were stored for total RNA extractions. Following RNA extractions, cDNA for each biological replicate is prepared and then transcript levels are determined for the target functional and housekeeping genes using q-PCR., Understanding Pcc/Pca cell densities, change in gene expression, AHL levels, and tissue maceration confirm the putative lactonase(s) are able to successfully attenuate bacterial pathogenesis.

### EXAMPLE 2

Quorum quenching lactonases from a wide variety of sources, e.g., an AHL from Ochrobactrum such as O. intermedium D-2, Bosea sp. such as strain F3-2, Rhizobiales, Rhodospirillales, Lysobacter such as L. enzymogenes, e.g., MomL, MomL(L254R), MomL(I144V), or MomL(V149A), Stenotrophomonas such as S. maltophilia, Rhodococcus such as L. erythropolis, Planococcus such as P. versutus, Enterobacter, Hyphomonas genus (Alphaproteobacteria), Lysinibacillus such as L. sphaericus or Geobacillus sp. may be employed in the methods and compositions, e.g., to prevent, inhibit or treat soft rot caused by bacterial infection in fruits, vegetables or ornamental plants, e.g., potatoes, cucumber, carrots, Chinese cabbage, peppers, onions, zucchini, or celery. For example, infection of fruit, vegetables of ornamental plants by Pectobacteria such as Pectobacteria atrosepticum or Pectobacteria carotovorum, Pseudomonas such as Pseudomonas aeruginosa, or Vibrio such as Vibrio coralliilyticus. Bacillus, Burkholderia, Pantoea, Enterobacter, Klebsiella, Leuconostoc or clostridia may cause soft rot.

Exemplary plants, or fruits or vegetables of those plants, that may be treated with the compositions include but are not limited to Chrysanthemum sp., Vanda sp., Amorphophallus konjac, Anubias barteri, Brassica rapa, Phalaenopsis aphrodite, Philodendron sp., Tagetes patula, Musa sp., Vanilla planifoliab, Cichorium intybus, Solanum tuberosum, Pyrus sp., Musa sp.; Hyacinthus orientalis, Solanum tuberosum, Musa sp., Oryza sativa, Zea mays, Ananas comosus, Phalaenopsis sp., Actinidia deliciosab, Cucurbita pepo, Ornithogalum dubium, Persea americana, Saccharum spp., Solanum tuberosum, Zantedeschia aethiopica, Helianthus annuus, Solanum melongena, Solanum tuberosum, Zantedeschia aethiopica, Beta vulgaris; Beta vulgaris. Brassica oleracea , Capsicum annuum, Cucumis sativus, Cucurbita pepo, Cynara cardunculusb, Nicotiana tabacum, Solanum lycopersicum, Solanum tuberosum, Carnegiea gigantea, Helianthus annuus, Hawthoria, Ipomoea batatas, Kalanchoe tubiflora, Lactuca sativa, Opuntia sp., Orostachys japonica, Orostachys malacophyll, Papaver somniferum, Peperomia obtusifolia, Peperomia caperata, Plectranthus australis, Pilea cadiereic, Pinellia ternate, Rheum rhabarbarum, Silybum marianum, Saintpaulia ionantha, Solanum lycopersicum, Solanum tuberosum, Spathiphyllum wallisii, Typhonium giganteum, Allium ampeloprasum, Allium cepa, Apium graveolens, Brassica oleracea, Brassica raga, Cichorium endivia, Cichorium intybus, Daucus carota, Ipomoea batatas, Petrosehnum crispum, Solanum tuberosum, Brassica oleracead, Eutrema japonicum, Ipomoea batatas, Solanum lycopersicum, Solanum melongena, or Erythrina indica.

### EXAMPLE 3

Pectobacterium carotovorum (NCPPB312) and Pectobacterium atrosepticum (SCRI1043), bacterial pathogens, utilize the N-acyl homoserine lactones as their secreted chemical language (quorum sensing), that plays a role in pathogenicity. It was determined if the various lactonases (putative quorum quenching enzymes) degrade their AHLs (3-oxo-hexanoyl homoserine lactone (3OC6HSL) and 3-oxo-octanoyl homoserine lactone (3OC8HSL)), thereby attenuating quorum sensing regulated physiology when grown in microbiological media and in plant tissues.

The enzymes clustered based upon amino acid similarity and predicted function into six different groups. These groups correspond to the following classifications of metal dependent hydrolases, beta-lactamase like proteins, hydroxyacylglutathione hydrolase, metallo-beta-lactamase superfamily proteins, Zn-dependent hydrolases, and phosphotriesterase like proteins. Based upon the similarities in AA composition, these enzymes in the potato assays were treated as the six groups instead of testing all enzymes.

### Pure Culture Tests

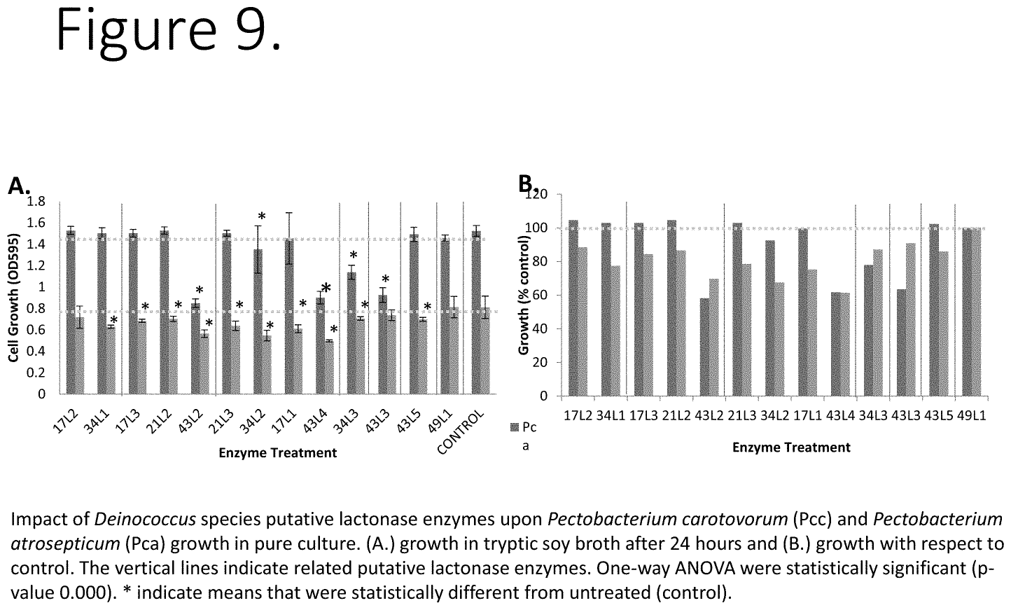

To determine how the putative QQ enzymes altered basic aspects of physiology and a quorum sensing controlled behavior, Pcc and Pca were grown in the presence of 5 uM concentrations of the various enzymes. Five enzymes significantly reduced growth of both pathogens (FIG. 9A), these enzymes belonged four of the different enzyme groups by function. However, there was not a uniform reduction in growth across the various enzyme groups or Pectobacterium species. Ten of various enzyme statistically reduced growth in Pcc as compared to five for Pca as compared to no enzyme treatment, which illustrates a potential species by species level effect of these enzymes. 12 of the enzymes treatment resulted in reduced growth in Pcc.

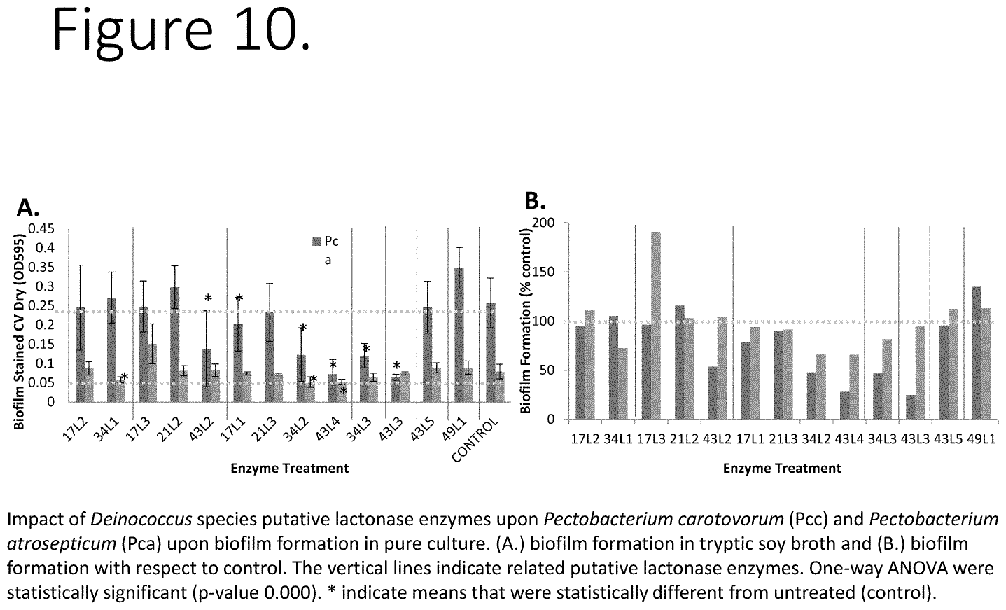

A similar trend was observed for the impacts upon biofilm formation (QS regulated response), which has been shown to be important in pathogenicity or infectivity. There were species level differences in how the enzymes negatively impacted biofilm formation (FIGS. 10A and 10B). Only two enzymes belonging to the groups, statistically reduced biofilm formation in both Pectobacterium species. Interestingly, Pca which was the stronger biofilm former was more impacted by exposure to these putative quorum quenching enzymes (lactonases). Six enzymes from three groups reduced biofilm formation in both species as compared to the control (FIG. 10B). However, three different groups decreased biofilm formation as compared to the bacteria growing alone (control). Interestingly, four of these enzymes belonged to one group, metal dependent hydrolases like proteins.

### Potato Assays

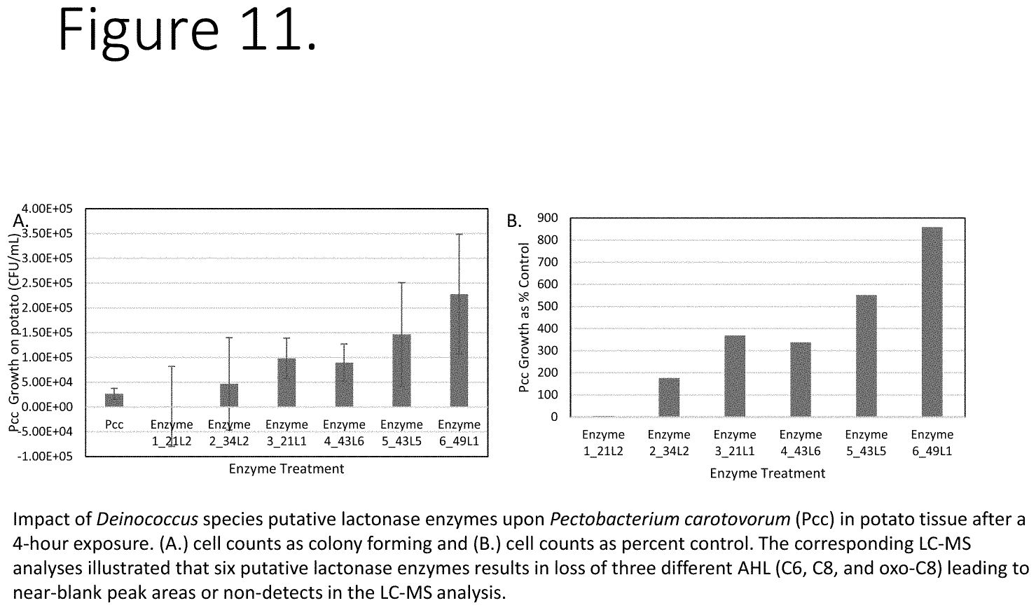

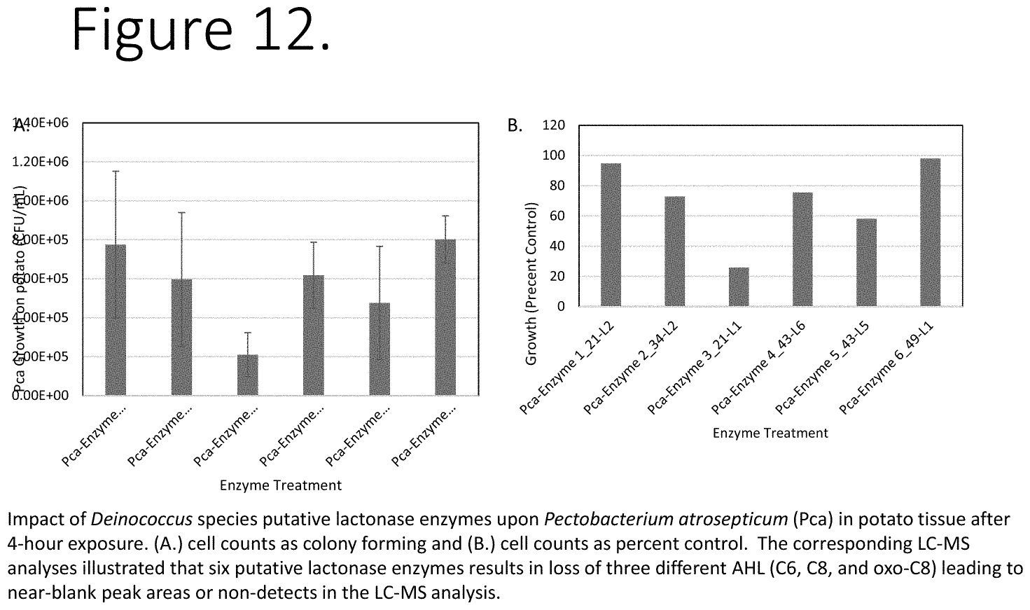

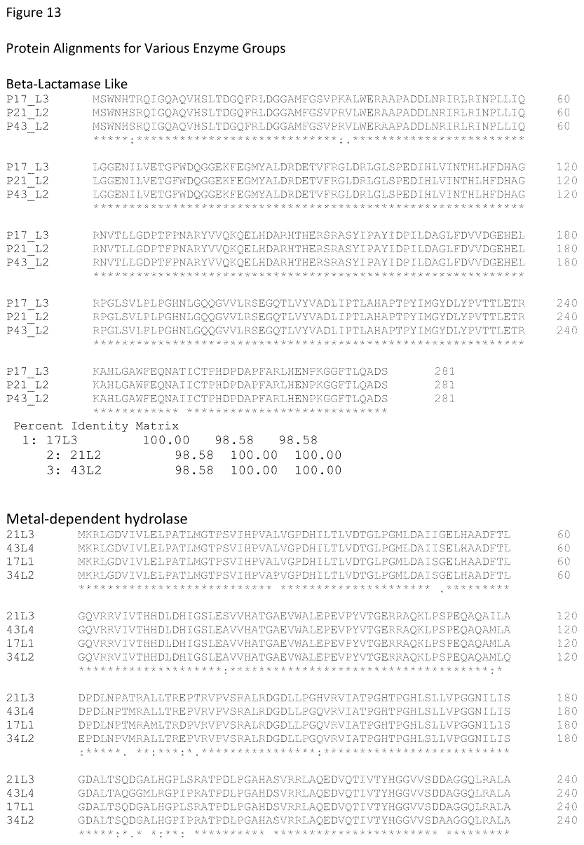

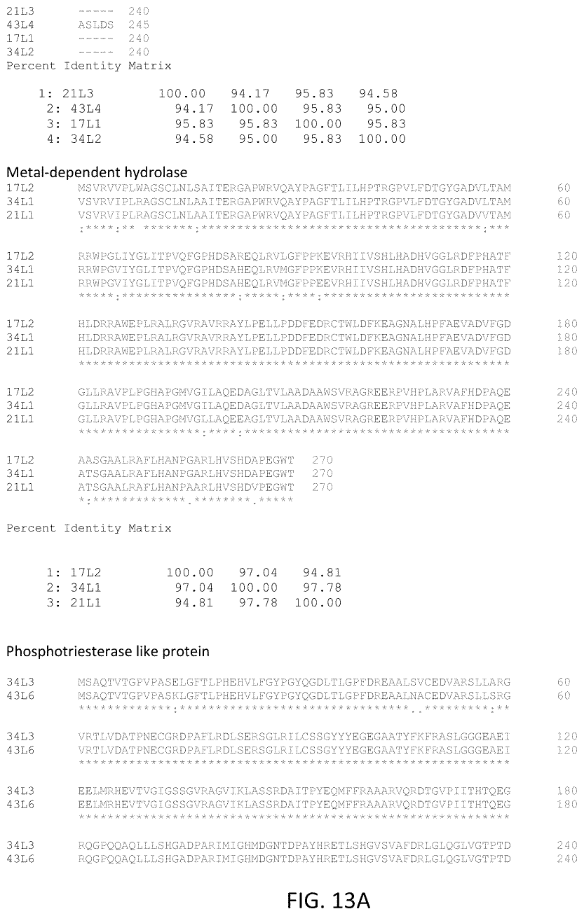

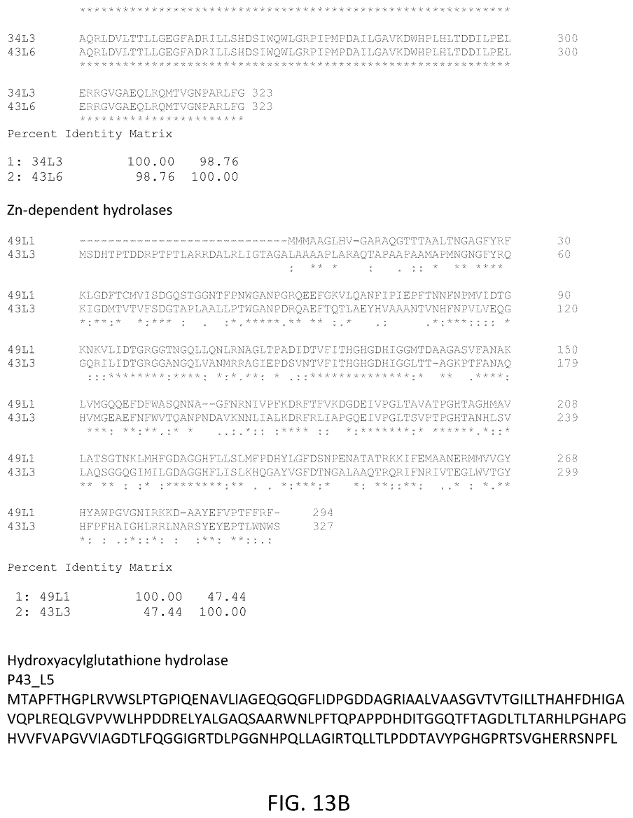

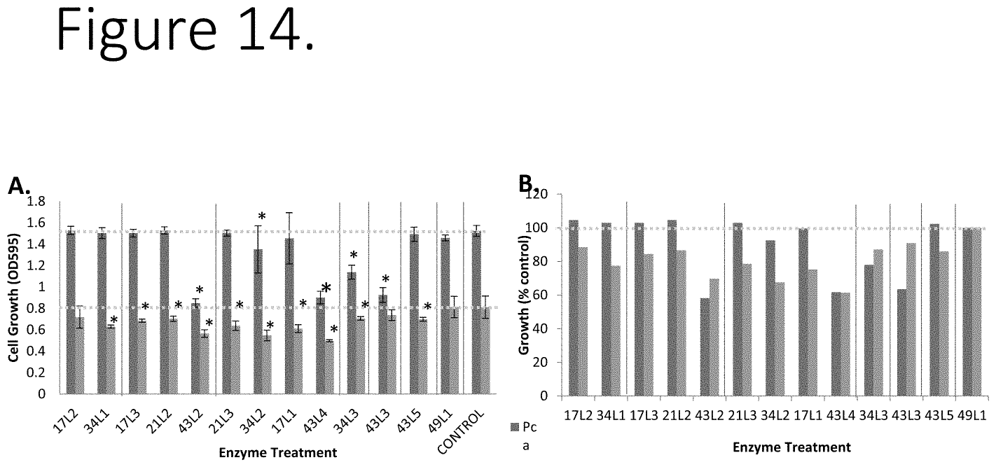

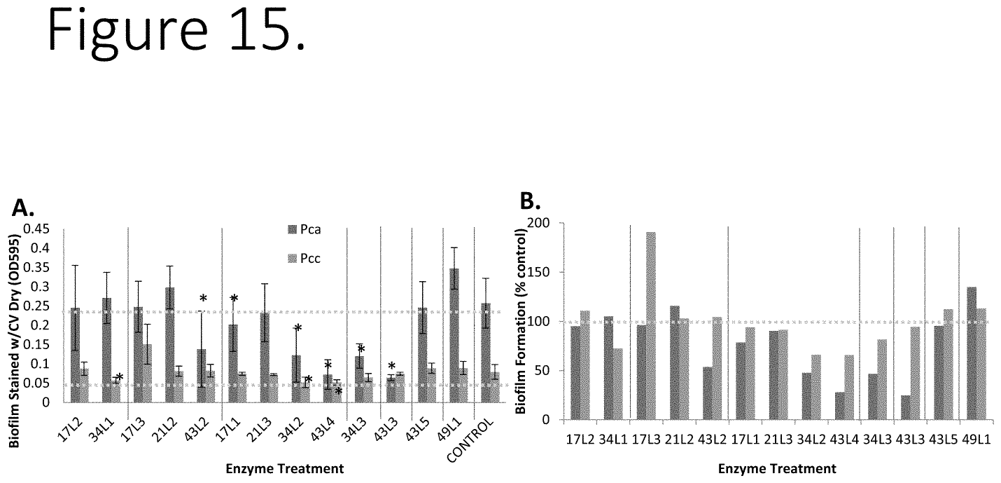

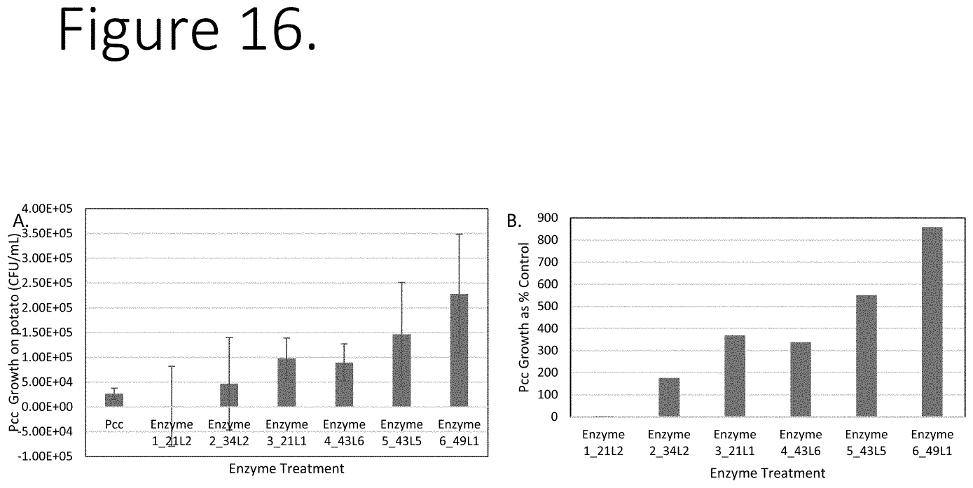

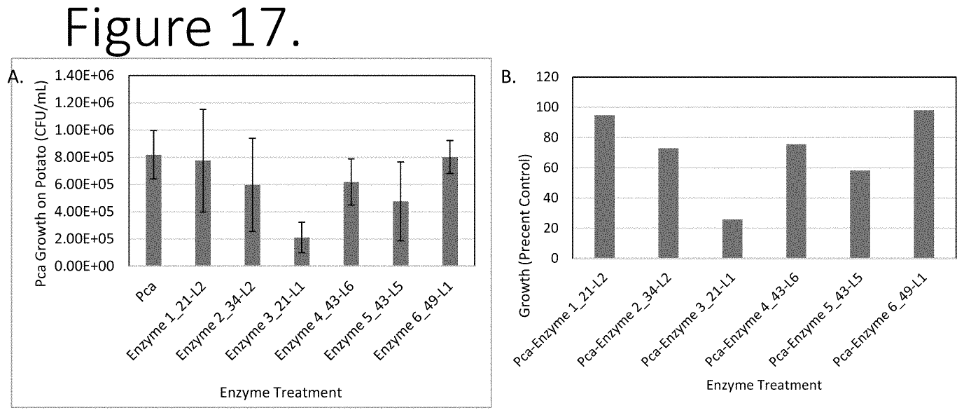

Lactonase function and impact upon plant growth tested using potatoes. A 4-hour time point was tested using an overnight culture, which allowed for detection of the AHLs of interest. The potato pieces were infected with the individually with the two pathogens and then growth and AHL levels were examined. Pcc grew during the 4-hour infection study (FIGS. 11A and 11B), however, there was loss of three different AHL (C6, C8, and oxo-C8) leading to near-blank peak areas or non-detects in the LC-MS analysis in these samples as compared to the untreated bacterium. A similar response was measured in Pca, however, the six enzyme groups inhibited growth, whereas only group 1 in Pcc impacted growth. A similar assay was testing AHL levels at 10- and 12-hour time point in Pcc, which again showed loss or below level of detection for the three AHLs assayed.

### REFERENCES

Barnard, A. M. and Salmond, G. P., 2007. Quorum sensing in Erwinia species. Analytical and bioanalytical chemistry, 387:415-423.
Charkowski, A., Blanco, C., Condemine, G., Expert, D., Franza, T., Hayes, C., Hugouvieux-Cotte-Pattat, N., Solanilla, E. L., Low, D., Moleleki, L. and Pirhonen, M., 2012. The role of secretion systems and small molecules in soft-rot Enterobacteriaceae pathogenicity. Annual review of phytopathology, 50: 425-449.
Crépin, A., Barbey, C., Beury-Cirou, A., Hélias, V., Taupin, L., Reverchon, S., Nasser, W., Faure, D., Dufour, A., Orange, N. and Feuilloley, M., 2012. Quorum sensing signaling molecules produced by reference and emerging soft-rot bacteria (Dickeya and Pectobacterium spp.). PLoS One, 7:e35176.
Dong, Y. H., Zhang, X. F., Xu, J. L. and Zhang, L. H., 2004. Insecticidal Bacillus thuringiensis silences Erwinia carotovora virulence by a new form of microbial antagonism, signal interference. Appl. Environ. Microbiol., 70:954-960.
Garge, S. S. and Nerurkar, A. S., 2017. Evaluation of quorum quenching Bacillus spp. for their biocontrol traits against Pectobacterium carotovorum subsp. carotovorum causing soft rot. Biocatalysis and agricultural biotechnology, 9:48-57. Graz University of Technology. Minimizing post-harvest food losses. ScienceDaily, 7 Nov. 2019. www.sciencedaily.com/releases/2019/11/191107111746.htm
Lee, D. H., Lim, J. A., Lee, J., Roh, E., Jung, K., Choi, M., Oh, C., Ryu, S., Yun, J. and Heu, S., 2013. Characterization of genes required for the pathogenicity of Pectobacterium carotovorum subsp. carotovorum Pcc21 in Chinese cabbage. Microbiology, 159:1487-96.
Mahmoudi, E., & Soleimani, R. (2019), “Host-induced gene silencing of Pectobacterium carotovorum quorum sensing gene enhances soft rot disease resistance in potato plants.” Archives of Phytopathology and Plant Protection, 1-29
Mueller-Spitz, S. R. and Crawford, K. D., 2014. Silver nanoparticle inhibition of polycyclic aromatic hydrocarbons degradation by Mycobacterium species RJGII-135. Letters in applied microbiology, 58(4), pp.330-337.
Põllumaa, L., Alamäe, T. and Mäe, A., 2012. Quorum sensing and expression of virulence in Pectobacteria. Sensors, 12:3327-3349.
Valente, R. S., Nadal-Jimenez, P., Carvalho, A. F., Vieira, F. J. and Xavier, K. B., 2017. Signal integration in quorum sensing enables cross-species induction of virulence in Pectobacterium wasabiae. MBio, 8: e00398-17.
Velásquez, A. C., Castroverde, C. D. M. and He, S. Y., 2018. Plant-pathogen warfare under changing climate conditions. Current Biology, 28: R619-R634

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method to prevent, inhibit or treat soft rot in a vegetable, fruit or ornamental plant, comprising: contacting the vegetable, the fruit or the ornamental plant with a composition comprising an effective amount of one or more isolated quorum quenching lactonases.

2. The method of claim 1 wherein the vegetable is a potato.

3. The method of claim 1 wherein the vegetable is a cucumber.

4. The method of claim 1 wherein the lactonase is a metal dependent hydrolase.

5. The method of claim 4 wherein the metal is zinc.

6. The method of claim 1 wherein the lactonase is a metallo-beta-lactamase.

7. The method of claim 1 wherein the vegetable or fruit is contacted with the composition.

8. The method of claim 1 wherein the vegetable is suspected of being infected with a pathogen.

9. The method of claim 8 wherein the pathogen comprises Dickeya or Pectobacterium.

10. The method of claim 8 wherein the pathogen comprises Erwinia or Pseudomonas.

11. The method of claim 1 wherein the lactonase is from Deinococcus.

12. The method of claim 1 wherein the lactonase has at least 80% amino acid sequence identity to one of SEQ ID Nos. 1-39.

13. The method of claim 1 wherein the contacting includes spraying, immersing, dripping a solution, or dusting with a powder.

14. A method to produce lactonases, comprising: expressing in a bacterial host cell a vector comprising an expression cassette comprising a heterologous promoter operably linked to an open reading frame encoding a bacterial lactonase; and isolating the expressed soluble lactonase.

15. The method of claim 14 wherein the lactonase is a metal dependent hydrolase.

16. The method of claim 14 wherein the lactonase is a metallo-beta-lactamase.

17. The method of claim 14 wherein lactonase is from Deinococcus.

18. The method of claim 14 wherein the lactonase has at least 80% amino acid sequence identity to one of SEQ ID Nos. 1-39.

19. An isolated host cell or a nucleic acid vector comprising a heterologous promoter operably linked to an open reading frame encoding a bacterial lactonase.

20. A composition comprising one or more isolated bacterial lactonases having at least 80% amino acid sequence identity to one of SEQ ID Nos. 1-39 and optionally a carrier.
